SEQUENCE LISTING (154 sequences)

SEQ ID NO: 1 - RNA, 23 nt, Artificial
  hsa-miR-135b
uauggcuuuucauuccuauguga

SEQ ID NO: 2 - RNA, 22 nt, Artificial
  hsa-miR-375
uuuguucguucggcucgcguga

SEQ ID NO: 3 - RNA, 22 nt, Artificial
  hsa-miR-381
uauacaagggcaagcucucugu

SEQ ID NO: 4 - RNA, 21 nt, Artificial
  hsa-miR-128
ucacagugaaccggucucuuu

SEQ ID NO: 5 - RNA, 22 nt, Artificial
  hsa-miR-199a-3p
acaguagucugcacauugguua

SEQ ID NO: 6 - RNA, 22 nt, Artificial
  hsa-miR-200b
uaauacugccugguaaugauga

SEQ ID NO: 7 - RNA, 23 nt, Artificial
  hsa-miR-339-5p
ucccuguccuccaggagcucacg

SEQ ID NO: 8 - RNA, 23 nt, Artificial
  hsa-miR-423-5p
ugaggggcagagagcgagacuuu

SEQ ID NO: 9 - RNA, 22 nt, Artificial
  hsa-miR-425*
aucgggaaugucguguccgccc

SEQ ID NO: 10 - RNA, 22 nt, Artificial
  hsa-miR-489
gugacaucacauauacggcagc

SEQ ID NO: 11 - RNA, 22 nt, Artificial
  hsa-miR-505*
gggagccaggaaguauugaugu

SEQ ID NO: 12 - RNA, 23 nt, Artificial
  hsa-miR-513-3p
uaaauuucaccuuucugagaagg

SEQ ID NO: 13 - RNA, 18 nt, Artificial
  hsa-miR-516a-3p
ugcuuccuuucagagggu

SEQ ID NO: 14 - RNA, 20 nt, Artificial
  hsa-miR-520d-5p
cuacaaagggaagcccuuuc

SEQ ID NO: 15 - RNA, 23 nt, Artificial
  hsa-miR-523
gaacgcgcuucccuauagagggu

SEQ ID NO: 16 - RNA, 22 nt, Artificial
  hsa-miR-643
acuuguaugcuagcucagguag

SEQ ID NO: 17 - RNA, 24 nt, Artificial
  hsa-miR-801
gauugcucugcgugcggaaucgac

SEQ ID NO: 18 - RNA, 22 nt, Artificial
  hsa-miR-99a
aacccguagauccgaucuugug

SEQ ID NO: 19 - RNA, 21 nt, Artificial
  hsa-let-7g*
cuguacaggccacugccuugc

SEQ ID NO: 20 - RNA, 22 nt, Artificial
  hsa-miR-1
uggaauguaaagaaguauguau

SEQ ID NO: 21 - RNA, 22 nt, Artificial
  hsa-miR-127-5p
cugaagcucagagggcucugau

SEQ ID NO: 22 - RNA, 22 nt, Artificial
  hsa-miR-132*
accguggcuuucgauuguuacu

SEQ ID NO: 23 - RNA, 23 nt, Artificial
  hsa-miR-136
acuccauuuguuuugaugaugga

SEQ ID NO: 24 - RNA, 22 nt, Artificial
  hsa-miR-136*
caucaucgucucaaaugagucu

SEQ ID NO: 25 - RNA, 23 nt, Artificial
  hsa-miR-145
guccaguuuucccaggaaucccu

SEQ ID NO: 26 - RNA, 23 nt, Artificial
  hsa-miR-18b
uaaggugcaucuagugcaguuag

SEQ ID NO: 27 - RNA, 21 nt, Artificial
  hsa-miR-190b
ugauauguuugauauuggguu

SEQ ID NO: 28 - RNA, 22 nt, Artificial
  hsa-miR-198
gguccagaggggagauagguuc

SEQ ID NO: 29 - RNA, 22 nt, Artificial
  hsa-miR-19a*
aguuuugcauaguugcacuaca

SEQ ID NO: 30 - RNA, 22 nt, Artificial
  hsa-miR-208b
auaagacgaacaaaagguuugu

SEQ ID NO: 31 - RNA, 22 nt, Artificial
  hsa-miR-210
cugugcgugugacagcggcuga

SEQ ID NO: 32 - RNA, 21 nt, Artificial
  hsa-miR-215
augaccuaugaauugacagac

SEQ ID NO: 33 - RNA, 22 nt, Artificial
  hsa-miR-22*
aguucuucaguggcaagcuuua

SEQ ID NO: 34 - RNA, 22 nt, Artificial
  hsa-miR-24-1*
ugccuacugagcugauaucagu

SEQ ID NO: 35 - RNA, 23 nt, Artificial
  hsa-miR-302d
uaagugcuuccauguuugagugu

SEQ ID NO: 36 - RNA, 22 nt, Artificial
  hsa-miR-335*
uuuuucauuauugcuccugacc

SEQ ID NO: 37 - RNA, 22 nt, Artificial
  hsa-miR-34a
uggcagugucuuagcugguugu

SEQ ID NO: 38 - RNA, 22 nt, Artificial
  hsa-miR-378*
cuccugacuccagguccugugu

SEQ ID NO: 39 - RNA, 22 nt, Artificial
  hsa-miR-382
gaaguuguucgugguggauucg

SEQ ID NO: 40 - RNA, 22 nt, Artificial
  hsa-miR-449b
aggcaguguauuguuagcuggc

SEQ ID NO: 41 - RNA, 22 nt, Artificial
  hsa-miR-486-5p
uccuguacugagcugccccgag

SEQ ID NO: 42 - RNA, 22 nt, Artificial
  hsa-miR-496
ugaguauuacauggccaaucuc

SEQ ID NO: 43 - RNA, 22 nt, Artificial
  hsa-miR-501-5p
aauccuuugucccugggugaga

SEQ ID NO: 44 - RNA, 22 nt, Artificial
  hsa-miR-518b
caaagcgcuccccuuuagaggu

SEQ ID NO: 45 - RNA, 21 nt, Artificial
  hsa-miR-518d-3p
caaagcgcuucccuuuggagc

SEQ ID NO: 46 - RNA, 21 nt, Artificial
  hsa-miR-518e
aaagcgcuucccuucagagug

SEQ ID NO: 47 - RNA, 22 nt, Artificial
  hsa-miR-542-3p
ugugacagauugauaacugaaa

SEQ ID NO: 48 - RNA, 22 nt, Artificial
  hsa-miR-551b*
gaaaucaagcgugggugagacc

SEQ ID NO: 49 - RNA, 23 nt, Artificial
  hsa-miR-567
aguauguucuuccaggacagaac

SEQ ID NO: 50 - RNA, 21 nt, Artificial
  hsa-miR-583
caaagaggaaggucccauuac

SEQ ID NO: 51 - RNA, 24 nt, Artificial
  hsa-miR-589*
ucagaacaaaugccgguucccaga

SEQ ID NO: 52 - RNA, 22 nt, Artificial
  hsa-miR-603
cacacacugcaauuacuuuugc

SEQ ID NO: 53 - RNA, 23 nt, Artificial
  hsa-miR-605
uaaaucccauggugccuucuccu

SEQ ID NO: 54 - RNA, 20 nt, Artificial
  hsa-miR-609
aggguguuucucucaucucu

SEQ ID NO: 55 - RNA, 22 nt, Artificial
  hsa-miR-615-3p
uccgagccugggucucccucuu

SEQ ID NO: 56 - RNA, 23 nt, Artificial
  hsa-miR-639
aucgcugcgguugcgagcgcugu

SEQ ID NO: 57 - RNA, 23 nt, Artificial
  hsa-miR-675
uggugcggagagggcccacagug

SEQ ID NO: 58 - RNA, 22 nt, Artificial
  hsa-miR-885-5p
uccauuacacuacccugccucu

SEQ ID NO: 59 - RNA, 22 nt, Artificial
  hsa-miR-130b
cagugcaaugaugaaagggcau

SEQ ID NO: 60 - RNA, 21 nt, Artificial
  hsa-miR-27b
uucacaguggcuaaguucugc

SEQ ID NO: 61 - RNA, 22 nt, Artificial
  hsa-miR-32
uauugcacauuacuaaguugca

SEQ ID NO: 62 - RNA, 21 nt, Artificial
  hsa-miR-323-3p
cacauuacacggucgaccucu

SEQ ID NO: 63 - RNA, 23 nt, Artificial
  hsa-miR-339-3p
ugagcgccucgacgacagagccg

SEQ ID NO: 64 - RNA, 24 nt, Artificial
  hsa-miR-362-5p
aauccuuggaaccuaggugugagu

SEQ ID NO: 65 - RNA, 23 nt, Artificial
  hsa-miR-425
aaugacacgaucacucccguuga

SEQ ID NO: 66 - RNA, 22 nt, Artificial
  hsa-miR-502-3p
aaugcaccugggcaaggauuca

SEQ ID NO: 67 - RNA, 21 nt, Artificial
  hsa-miR-126*
cauuauuacuuuugguacgcg

SEQ ID NO: 68 - RNA, 22 nt, Artificial
  hsa-miR-130a
cagugcaauguuaaaagggcau

SEQ ID NO: 69 - RNA, 22 nt, Artificial
  hsa-miR-143*
ggugcagugcugcaucucuggu

SEQ ID NO: 70 - RNA, 22 nt, Artificial
  hsa-miR-144*
ggauaucaucauauacuguaag

SEQ ID NO: 71 - RNA, 22 nt, Artificial
  hsa-miR-145*
ggauuccuggaaauacuguucu

SEQ ID NO: 72 - RNA, 22 nt, Artificial
  hsa-miR-181a*
accaucgaccguugauuguacc

SEQ ID NO: 73 - RNA, 21 nt, Artificial
  hsa-miR-188-3p
cucccacaugcaggguuugca

SEQ ID NO: 74 - RNA, 22 nt, Artificial
  hsa-miR-193a-3p
aacuggccuacaaagucccagu

SEQ ID NO: 75 - RNA, 22 nt, Artificial
  hsa-miR-19b-2*
aguuuugcagguuugcauuuca

SEQ ID NO: 76 - RNA, 22 nt, Artificial
  hsa-miR-202*
uuccuaugcauauacuucuuug

SEQ ID NO: 77 - RNA, 22 nt, Artificial
  hsa-miR-208
auaagacgagcaaaaagcuugu

SEQ ID NO: 78 - RNA, 21 nt, Artificial
  hsa-miR-220
ccacaccguaucugacacuuu

SEQ ID NO: 79 - RNA, 23 nt, Artificial
  hsa-miR-221
agcuacauugucugcuggguuuc

SEQ ID NO: 80 - RNA, 24 nt, Artificial
  hsa-miR-29b-1*
gcugguuucauauggugguuuaga

SEQ ID NO: 81 - RNA, 22 nt, Artificial
  hsa-miR-29c*
ugaccgauuucuccugguguuc

SEQ ID NO: 82 - RNA, 23 nt, Artificial
  hsa-miR-302a
uaagugcuuccauguuuugguga

SEQ ID NO: 83 - RNA, 23 nt, Artificial
  hsa-miR-324-5p
cgcauccccuagggcauuggugu

SEQ ID NO: 84 - RNA, 22 nt, Artificial
  hsa-miR-331-5p
cuagguauggucccagggaucc

SEQ ID NO: 85 - RNA, 22 nt, Artificial
  hsa-miR-33a*
caauguuuccacagugcaucac

SEQ ID NO: 86 - RNA, 21 nt, Artificial
  hsa-miR-376a
aucauagaggaaaauccacgu

SEQ ID NO: 87 - RNA, 22 nt, Artificial
  hsa-miR-450b-5p
uuuugcaauauguuccugaaua

SEQ ID NO: 88 - RNA, 22 nt, Artificial
  hsa-miR-519e
aagugccuccuuuuagaguguu

SEQ ID NO: 89 - RNA, 21 nt, Artificial
  hsa-miR-524-3p
gaaggcgcuucccuuuggagu

SEQ ID NO: 90 - RNA, 22 nt, Artificial
  hsa-miR-548d-5p
aaaaguaauugugguuuuugcc

SEQ ID NO: 91 - RNA, 22 nt, Artificial
  hsa-miR-550*
ugucuuacucccucaggcacau

SEQ ID NO: 92 - RNA, 19 nt, Artificial
  hsa-miR-564
aggcacggugucagcaggc

SEQ ID NO: 93 - RNA, 19 nt, Artificial
  hsa-miR-566
gggcgccugugaucccaac

SEQ ID NO: 94 - RNA, 23 nt, Artificial
  hsa-miR-582-5p
uuacaguuguucaaccaguuacu

SEQ ID NO: 95 - RNA, 21 nt, Artificial
  hsa-miR-587
uuuccauaggugaugagucac

SEQ ID NO: 96 - RNA, 23 nt, Artificial
  hsa-miR-623
aucccuugcaggggcuguugggu

SEQ ID NO: 97 - RNA, 21 nt, Artificial
  hsa-miR-628-3p
ucuaguaagaguggcagucga

SEQ ID NO: 98 - RNA, 22 nt, Artificial
  hsa-miR-649
aaaccuguguuguucaagaguc

SEQ ID NO: 99 - RNA, 23 nt, Artificial
  hsa-miR-672
ugagguugguguacuguguguga

SEQ ID NO: 100 - RNA, 23 nt, Artificial
  hsa-miR-708
aaggagcuuacaaucuagcuggg

SEQ ID NO: 101 - RNA, 22 nt, Artificial
  hsa-miR-98
ugagguaguaaguuguauuguu

SEQ ID NO: 102 - RNA, 22 nt, Artificial
  hsa-let-7a
ugagguaguagguuguauaguu

SEQ ID NO: 103 - RNA, 22 nt, Artificial
  hsa-miR-126
ucguaccgugaguaauaaugcg

SEQ ID NO: 104 - RNA, 22 nt, Artificial
  hsa-miR-141
uaacacugucugguaaagaugg

SEQ ID NO: 105 - RNA, 22 nt, Artificial
  hsa-miR-148a
ucagugcacuacagaacuuugu

SEQ ID NO: 106 - RNA, 22 nt, Artificial
  hsa-miR-148b
ucagugcaucacagaacuuugu

SEQ ID NO: 107 - RNA, 22 nt, Artificial
  hsa-miR-361-5p
uuaucagaaucuccagggguac

SEQ ID NO: 108 - RNA, 21 nt, Artificial
  hsa-miR-625
agggggaaaguucuauagucc

SEQ ID NO: 109 - RNA, 22 nt, Artificial
  hsa-miR-630
aguauucuguaccagggaaggu

SEQ ID NO: 110 - RNA, 22 nt, Artificial
  hsa-miR-766
acuccagccccacagccucagc

SEQ ID NO: 111 - RNA, 22 nt, Artificial
  hsa-let-7b
ugagguaguagguugugugguu

SEQ ID NO: 112 - RNA, 22 nt, Artificial
  hsa-let-7c
ugagguaguagguuguaugguu

SEQ ID NO: 113 - RNA, 22 nt, Artificial
  hsa-let-7e
ugagguaggagguuguauaguu

SEQ ID NO: 114 - RNA, 22 nt, Artificial
  hsa-let-7g
ugagguaguaguuuguacaguu

SEQ ID NO: 115 - RNA, 22 nt, Artificial
  hsa-miR-100
aacccguagauccgaacuugug

SEQ ID NO: 116 - RNA, 22 nt, Artificial
  hsa-miR-125b
ucccugagacccuaacuuguga

SEQ ID NO: 117 - RNA, 22 nt, Artificial
  hsa-miR-139-5p
ucuacagugcacgugucuccag

SEQ ID NO: 118 - RNA, 22 nt, Artificial
  hsa-miR-146b-5p
ugagaacugaauuccauaggcu

SEQ ID NO: 119 - RNA, 23 nt, Artificial
  hsa-miR-181a
aacauucaacgcugucggugagu

SEQ ID NO: 120 - RNA, 22 nt, Artificial
  hsa-miR-181a-2*
accacugaccguugacuguacc

SEQ ID NO: 121 - RNA, 22 nt, Artificial
  hsa-miR-186
caaagaauucuccuuuugggcu

SEQ ID NO: 122 - RNA, 21 nt, Artificial
  hsa-miR-188-5p
caucccuugcaugguggaggg

SEQ ID NO: 123 - RNA, 23 nt, Artificial
  hsa-miR-191
caacggaaucccaaaagcagcug

SEQ ID NO: 124 - RNA, 22 nt, Artificial
  hsa-miR-193b
aacuggcccucaaagucccgcu

SEQ ID NO: 125 - RNA, 22 nt, Artificial
  hsa-miR-23a*
gggguuccuggggaugggauuu

SEQ ID NO: 126 - RNA, 22 nt, Artificial
  hsa-miR-26a
uucaaguaauccaggauaggcu

SEQ ID NO: 127 - RNA, 22 nt, Artificial
  hsa-miR-30e*
cuuucagucggauguuuacagc

SEQ ID NO: 128 - RNA, 23 nt, Artificial
  hsa-miR-335
ucaagagcaauaacgaaaaaugu

SEQ ID NO: 129 - RNA, 21 nt, Artificial
  hsa-miR-342-5p
aggggugcuaucugugauuga

SEQ ID NO: 130 - RNA, 22 nt, Artificial
  hsa-miR-365
uaaugccccuaaaaauccuuau

SEQ ID NO: 131 - RNA, 22 nt, Artificial
  hsa-miR-505
cgucaacacuugcugguuuccu

SEQ ID NO: 132 - RNA, 22 nt, Artificial
  hsa-miR-532-3p
ccucccacacccaaggcuugca

SEQ ID NO: 133 - RNA, 22 nt, Artificial
  hsa-miR-532-5p
caugccuugaguguaggaccgu

SEQ ID NO: 134 - RNA, 22 nt, Artificial
  hsa-miR-576-3p
aagauguggaaaaauuggaauc

SEQ ID NO: 135 - RNA, 23 nt, Artificial
  hsa-miR-579
uucauuugguauaaaccgcgauu

SEQ ID NO: 136 - RNA, 21 nt, Artificial
  hsa-miR-629
uggguuuacguugggagaacu

SEQ ID NO: 137 - RNA, 19 nt, Artificial
  hsa-miR-645
ucuaggcugguacugcuga

SEQ ID NO: 138 - RNA, 23 nt, Artificial
  hsa-miR-92a-1*
agguugggaucgguugcaaugcu

SEQ ID NO: 139 - RNA, 22 nt, Artificial
  hsa-miR-16-1*
ccaguauuaacugugcugcuga

SEQ ID NO: 140 - RNA, 23 nt, Artificial
  hsa-miR-18a
uaaggugcaucuagugcagauag

SEQ ID NO: 141 - RNA, 20 nt, Artificial
  hsa-miR-202
agagguauagggcaugggaa

SEQ ID NO: 142 - RNA, 22 nt, Artificial
  hsa-miR-203
gugaaauguuuaggaccacuag

SEQ ID NO: 143 - RNA, 21 nt, Artificial
  hsa-miR-212
uaacagucuccagucacggcc

SEQ ID NO: 144 - RNA, 22 nt, Artificial
  hsa-miR-27a*
agggcuuagcugcuugugagca

SEQ ID NO: 145 - RNA, 22 nt, Artificial
  hsa-miR-29a*
acugauuucuuuugguguucag

SEQ ID NO: 146 - RNA, 23 nt, Artificial
  hsa-miR-330-3p
gcaaagcacacggccugcagaga

SEQ ID NO: 147 - RNA, 22 nt, Artificial
  hsa-miR-34a*
caaucagcaaguauacugcccu

SEQ ID NO: 148 - RNA, 22 nt, Artificial
  hsa-miR-380*
ugguugaccauagaacaugcgc

SEQ ID NO: 149 - RNA, 22 nt, Artificial
  hsa-miR-483-5p
aagacgggaggaaagaagggag

SEQ ID NO: 150 - RNA, 21 nt, Artificial
  hsa-miR-518f
gaaagcgcuucucuuuagagg

SEQ ID NO: 151 - RNA, 20 nt, Artificial
  hsa-miR-572
guccgcucggcgguggccca

SEQ ID NO: 152 - RNA, 22 nt, Artificial
  has-miR-597
ugugucacucgaugaccacugu

SEQ ID NO: 153 - RNA, 25 nt, Artificial
  has-miR-638
agggaucgcgggcggguggcggccu

SEQ ID NO: 154 - RNA, 19 nt, Artificial
  hsa-miR-626
agcugucugaaaaugucuu

(19) United States
(12) Patent Application Publication
(10) Pub. No.: US 2013/0165497 A1
(43) Pub. Date: Jun. 27, 2013

(54) MONITORING OF IMMUNE SYSTEM USING PERIPHERAL BLOOD MICRO-RNA EXPRESSION PROFILE ANALYSIS AND USES THEREOF

(71) Applicants: Sergio Abrignanai, Serre Di Rapolano Siena (SI) (IT); Raffaele De Francesco, Milano (MI) (IT); Massimiliano Pagani, Trescore Balneario (IT)

(72) Inventors: Sergio Abrignanai, Serre Di Rapolano Siena (SI) (IT); Raffaele De Francesco, Milano (MI) (IT); Massimiliano Pagani, Trescore Balneario (IT)

(73) Assignee: ISTITUTO NAZIONALE DI GENETICA MOLECOLARE - INGM, Milano (IT)

(21) Appl. No.: 13/703,730

(22) PCT Filed: Jun. 15, 2011
(86) PCT No.: PCT/IB2011/052599

§ 371 Date: Jan. 15, 2013

(30) Foreign Application Priority Data
Jun. 16, 2010 (IT) MI2010A001089

(51) Int. Cl. C12Q 1/68; A61K 31/7105

(52) U.S. Cl. CPC C12Q 1/686; C12Q 1/6837; A61K 31/7105
USPC 514/44 A; 435/6.12; 506/9

(57) ABSTRACT

The present invention relates to a method for monitoring the immune system of an individual, which comprises measuring, preferably by quantitative RT-PCR, the expression level of at least one microRNA (miRNA) gene product in a peripheral blood sample or in a biological fluid sample, and comparing said measured expression level with a reference level. In particular, the at least one miRNA gene product, which the method of the invention measures, is expressed by lymphocyte populations of an individual, in particular by naive CD4+T, TH1, TH2 and TH17 lymphocytes. The method of the invention is useful for the diagnosis, prognosis, prevention, control and/or the treatment of a pathological condition caused by or associated with an immune system dysfunction. Moreover, the method of the present invention is useful for monitoring, in an individual, the evolution of conditions mediated by the immune system, such as the response to a vaccination.

The present invention relates to a method for monitoring the immune system of an individual in pathological conditions caused by or associated with an immune system dysfunction.

In particular, said pathological conditions can be immunodeficiencies, neoplasia of the immune system or immune-mediated pathologies, for example an allergy condition or an autoimmune pathology.

Furthermore, the method of the present invention can be used for monitoring or “follow-up” of a vaccination.

The immune system has the function of protecting the body from assault by foreign agents, called antigens.

The defense function is carried out by means of specialized cells, defined as immunocompetent, which are scattered and circulating and organized in primary and secondary lymphoid organs.

The cellular elements of the immune system are:
T, B and Natural Killer (NK) lymphocytes;
macrophages (which derive from the circulating blood monocytes that generate the large family of antigen presenting cells, or “APC”).

Other immunocompetent cells circulating in the blood are neutrophil granulocytes, eosinophil granulocytes and basophil granulocytes.

This fine, efficient defense system of the body can at times be functionally altered until resulting, in some cases, in a compromise of the body.

In immunodeficiencies, for example, one observes an increased susceptibility to infections and several neoplasia due to the absence or inefficiency of some parts of the immune system. The absence or inefficiency of the immune system can be congenital or acquired pharmacologically or through infection (as in Acquired Immunodeficiency Syndrome).

Insofar as regards immune-mediated pathologies, they can be caused by or associated with functional anomalies of the immune system which manifest themselves with an “unbalancing” of its activity toward a specific cell line. What may occur is an uncontrolled activity of the cell line concerned in its maturation phases and, consequently, an impairment of its effector functions.

Hypersensitivity reactions and allergies represent particular, often transient, immune-mediated clinical conditions, likewise correlated with an immune system dysfunction.

Such conditions are characterized by an exaggerated activity of the immune system in response to innocuous antigens, defined as allergens. The most common form of such a dysfunction, so-called allergy in the strict sense, is mediated by IgE and associated with the activation of mastocytes.

Among the other pathological conditions which involve an immune system dysfunction, also of particular interest are autoimmune diseases, that are, pathologies in which the immune response is directed against “self” antigens, i.e. toward normal constituents of the body. The latter represents a physiological mechanism of the immune system designed to produce minimal quantities of autoantibodies useful for maintaining and improving the body's capacity to discriminate between what is “self” and “non-self”, i.e. between the elements belonging to it versus the ones foreign to it.

This particular capacity of the immune system is called tolerance.

Autoimmune diseases comprise the group of pathologies which are correlated with the alteration of this fine mechanism and are characterized by a substantial production of antibodies capable of striking individual organs or of triggering systemic diseases which, in extreme cases, are capable of completely compromising several functions of an individual. Examples of autoimmune diseases are systemic lupus erythematosus, rheumatoid arthritis, ankylosing spondylitis, multiple sclerosis, type 1 diabetes mellitus and psoriatic arthritis.

The above-mentioned immune system dysfunctions can affect lymphocyte populations, T lymphocytes in particular.

T lymphocytes are cells that originate from bone marrow, but they mature in the thymus, where they acquire both their specific functional capacity, and the concept of “self”. Once mature, T lymphocytes leave the gland and are to be found in peripheral blood and inside lymphoid tissues. They express the membrane protein CD4 or CD8, in a mutually exclusive manner.

The T cells which express CD4 are generically called T helper lymphocytes (T_{H}) and generally represent the cells that regulate adaptive immune responses and inflammatory diseases. These cells can be divided into various main categories, according to their function, response to different cytokines and capacity to secrete cytokines.

The current opinion is that T_{H} cells originate as cell precursors that produce Interleukin-2 (IL-2). As a result of the initial stimulation, these cells are transformed into naive CD4⁺ T (T_{H}0) cells, which have the capacity to secrete various cytokines, including interferon gamma (IFN-γ), IL-2, IL-4, IL-5 and IL-10.

Based on the cytokine available, T_{H}0 cells can give rise to different T_{H} cells.

In particular, IFN-γ and IL-12 promote the development of T_{H}1 cells, which serve to regulate cellular immunity. Thanks to the characteristic production of IFN-γ and the activation of macrophages, these cells mediate protection against intracellular pathogens and are moreover responsible for delayed hypersensitivity responses.

The presence of IL-4 and IL-10, on the other hand, promotes the differentiation of T_{H}2 cells capable of modulating humoural immunity and allergic responses. Moreover, through the production of IL-4, IL-5 and IL-13, these cells contribute to protection against extracellular parasites. Recently, a new line of T helper lymphocytes has been isolated and characterized; it is distinct from T_{H}1 and T_{H}2 and defined as T_{H}17 because of its capacity to secrete IL-17. This line of T lymphocytes plays a fundamental role in autoimmunity and inflammation. The differentiation of T_{H}17 lymphocytes from undifferentiated precursors is guided, during the immune responses, by cytokines and specific transcriptional factors.

In particular, it has been demonstrated that the differentiation of these cells in vitro is inhibited by the presence of T_{H}1 and T_{H}2 lymphocytes. In light of the key role of T_{H}17 cells in autoimmunity and inflammation, it is believed that, under normal conditions, there exists a fine mechanism which controls T_{H}17 cells by repressing them. This mechanism seems to be mediated by cytokines involved in the biology of T_{H}1 and T_{H}2 lymphocytes.

At present, the most accredited therapies for fighting immunodeficiencies are essentially pharmacological therapies. For example, the therapies used to fight AIDS are based on antiretroviral drugs belonging to different pharmacological classes, each characterized by a different mechanism of action. None of these drugs are capable of killing the virus, but rather they act by blocking the replication thereof. Such drugs, therefore, are not curative at present and the patients undergoing treatment must always be considered potentially infectious, even if they have an undetectable blood viral load.

Furthermore, pharmacological therapy is often complicated by the difficult tolerability of the drugs, which can cause side effects requiring the suspension thereof and entail a considerable effort on the patient's part in order to comply with the dosages and the methods of intake. Finally, the drugs used have difficulty in penetrating into various regions of the body, a difficulty which prevents them from attacking the virus in these regions; such difficulty is also accompanied by a possible onset of resistance, which renders the action of the drugs used ineffective.

In general, the current therapeutic approach toward immunodeficiency follows the motto “Hit early, hit hard”; that is, it is preferred to begin the therapy earlier than was done in the past. The rationale of this strategy consists in beginning the therapy as soon as possible so as to block viral replication when the immune system is still efficient and thus able to fully recover its functions. This avoids the possible occurrence of mutations in the viral population which could induce resistance to the therapy itself.

The possibility of monitoring the immune system and the functionalities and responses thereof could represent a valid clinical tool which could make it possible both to identify the most timely moment at which to undertake the pharmacological therapy against the immunodeficiency and follow the course of the disease during and after the treatment.

Insofar as allergies are concerned, on the other hand, the specific therapeutic approaches available provide for the administration of drugs capable of blocking antibodies or of antihistaminic drugs. In the most acute forms, cortisone drugs capable of blocking the immune system in a more decided manner are administered, but they simultaneously cause greater toxicity.

In general, what is carried out therapeutically is a veritable vaccine-based immunotherapy, by means of which an attempt is made to remedy the error of the immune system by inducing a state of being “accustomed” to the presence of allergenic substances. This therapeutic approach, however, has some limiting aspects, which on the one hand regard the fact that an individual allergic to one substance may become allergic to others; on the other hand, in many cases the immune system continues in its error anyway.

As far as autoimmune pathologies are concerned, the implementable therapeutic approaches are often closely tied to the individual pathologies and are usually limited solely to alleviating the discomfort associated with them rather than eradicating the cause that triggers them.

Therapeutic treatment against autoimmune pathologies often involves controlling, by means of drugs, the various physiological aspects of the immune response, such as, for example, inflammation.

The most accredited drugs are steroids, or else immunosuppressive drugs can be used. The administration of steroid drugs can give rise to many adverse side effects; however, the practice is implemented all the same on the basis of the balance between benefits and adverse side effects.

Immunosuppressive drugs, on the other hand, inhibit the division of cells, including cells that do not belong to the immune system and thus in this case as well the effect can prove very dangerous.

In view of the limited therapeutic options which can be implemented against pathological conditions caused by or associated with an immune system dysfunction, there is a strongly felt need to identify new therapeutic methods suitable for preventing, controlling and/or treating said pathological conditions, and for improving the discomfort associated with them and hence the patient's quality of life. At the same time, there is also a strongly felt need to have methods for evaluating the risk of compromising the functionality of the immune system, or methods for monitoring the effectiveness of a therapy designed to treat a pathological condition caused by or associated with an immune system dysfunction or for monitoring the evolution of conditions mediated by the immune system, for example for monitoring a response to a vaccination.

Such therapeutic methods would prove useful above all for improving human health; moreover, they would contribute to dampening the social costs of health.

The above-described technical problems are solved by a method for monitoring the immune system of an individual as outlined in the appended claims.

The present invention relates to a method for monitoring the immune system (in particular, the functionality of the immune system) of an individual, preferably when this individual is affected by a pathological condition caused by or associated with an immune system dysfunction. The method of the invention can also be used to monitor the evolution of conditions mediated by the immune system (for example, to monitor the response to a vaccination).

Said method for monitoring the immune system (in particular the functionality thereof) of an individual is useful for the diagnosis, prognosis, prevention, control and/or treatment of a pathological condition caused by or associated with an immune system dysfunction.

Moreover, said method for monitoring the immune system of an individual is useful for evaluating the risk of the functionality of the immune system itself being compromised, or for monitoring the effectiveness of a therapy designed to treat a pathological condition caused by or associated with an immune system dysfunction in an individual, or for monitoring, in an individual, the evolution of conditions mediated by the immune system, such as, for example, the response to a vaccination.

The method according to the present invention comprises measuring, preferably by quantitative RT-PCR, the expression level of at least one gene product of a microRNA (miRNA), preferably the expression level of at least two miRNA gene products, in a sample of peripheral blood or in a sample of biological fluid, and comparing said expression level measured with a reference level.

In particular, said at least one miRNA gene product is expressed by lymphocyte populations, preferably by T lymphocytes, more preferably by T helper lymphocytes which express the membrane protein CD4.

For example, the T helper lymphocytes which express the protein CD4 are naive CD4⁺ T, T_{H}1, T_{H}2 and T_{H}17.

An alteration in the expression levels of the miRNA gene product in a sample of the test subject, when compared to a control sample or level, is indicative of the fact that in the subject there exists an immune system dysfunction or there is an increased risk that an immune system dysfunction will occur. This method is thus useful for the diagnosis or prevention of pathological conditions caused by or associated with an immune system dysfunction.

Furthermore, an alteration in the expression levels of the miRNA gene product in a sample of the test subject, when compared to a control sample or level, is indicative of the effectiveness, evolution and outcome of a therapy against a pathological condition caused by or associated with a dysfunction of an individual's immune system.

An alteration in the expression levels of the miRNA gene product in a sample of the test subject, when compared to a control sample or level, is also indicative of the evolution of a pathological condition and hence of its prognosis.

Finally, an alteration in the expression levels of the miRNA gene product in a sample of the test subject, when compared to a control sample or level, is indicative of the follow-up of a vaccination in an individual who underwent said vaccination.

Further characteristics and advantages of the method according to the present invention will be more apparent from the experimental results illustrated in the appended figures, in which:

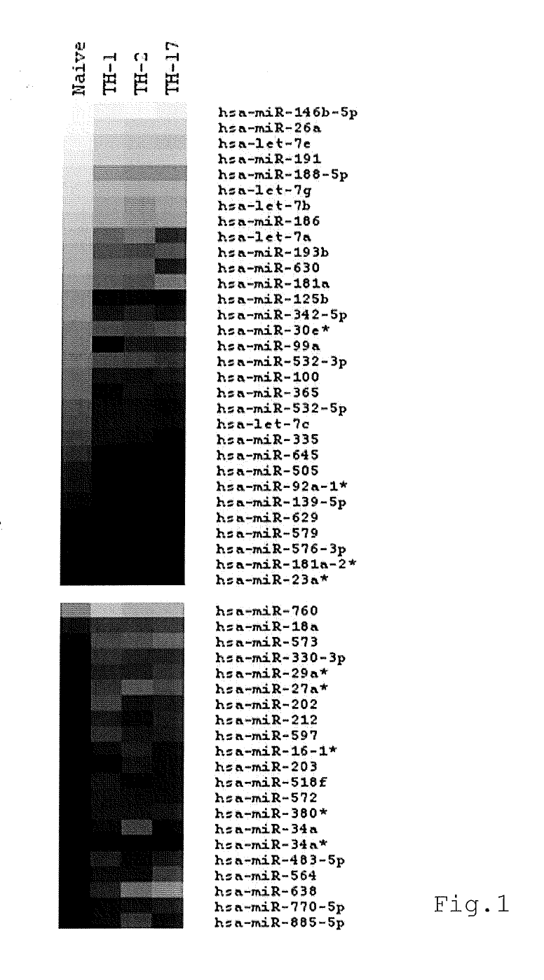

FIG. 1 shows a graphic representation by colour gradient (heatmap) of the DCt values (Ct: cycle threshold) for the overexpressed miRNAs (top panel) and for the underexpressed miRNAs (bottom panel) in the naive CD4⁺ T lymphocyte population, as compared to the T_{H}1, T_{H}2 and T_{H}17 lymphocyte populations.

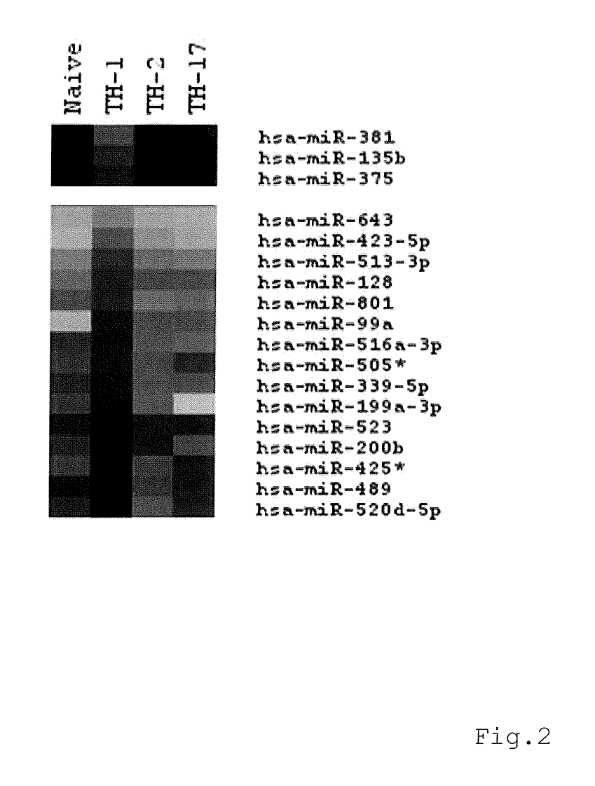

FIG. 2 shows a graphic representation by colour gradient (heatmap) of the DCt values (Ct: cycle threshold) for the overexpressed miRNAs (top panel) and for the underexpressed miRNAs (bottom panel) in the T_{H}1 lymphocyte population, as compared to the naive CD4⁺ T, T_{H}2 and T_{H}17 lymphocyte populations.

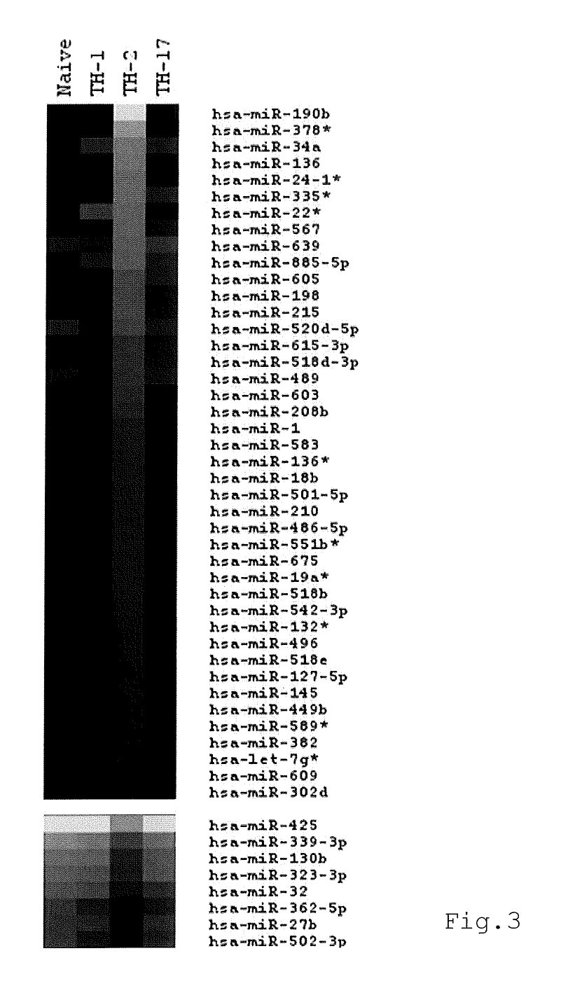

FIG. 3 shows a graphic representation by colour gradient (heatmap) of the DCt values (Ct: cycle threshold) for the overexpressed miRNAs (top panel) and for the underexpressed miRNAs (bottom panel) in the T_{H}2 lymphocyte population, as compared to the naive CD4⁺ T, T_{H}1 and T_{H}17 lymphocyte populations.

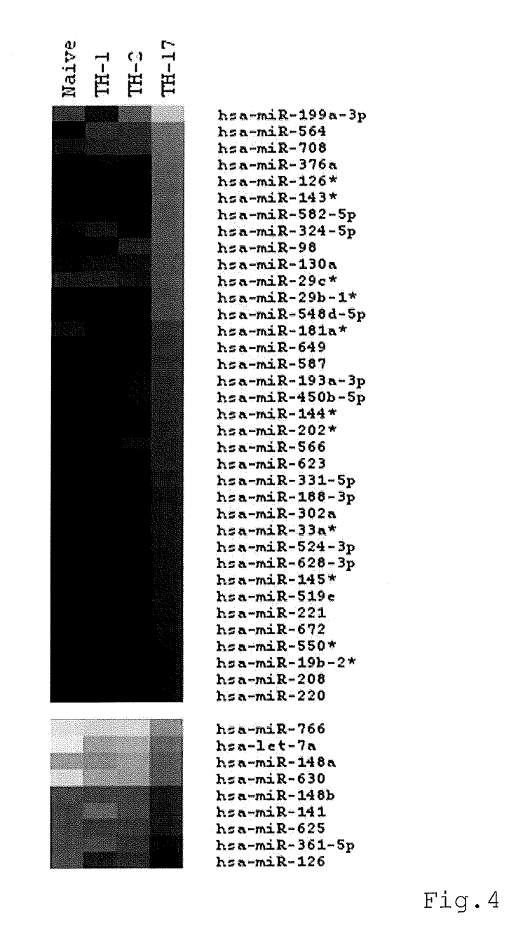

FIG. 4 shows a graphic representation by colour gradient (heatmap) of the DCt values (Ct: cycle threshold) for the overexpressed miRNAs (top panel) and for the underexpressed miRNAs (bottom panel) in the T_{H}17 lymphocyte population, as compared to the naive CD4⁺ T, T_{H}1 and T_{H}2 lymphocyte populations.

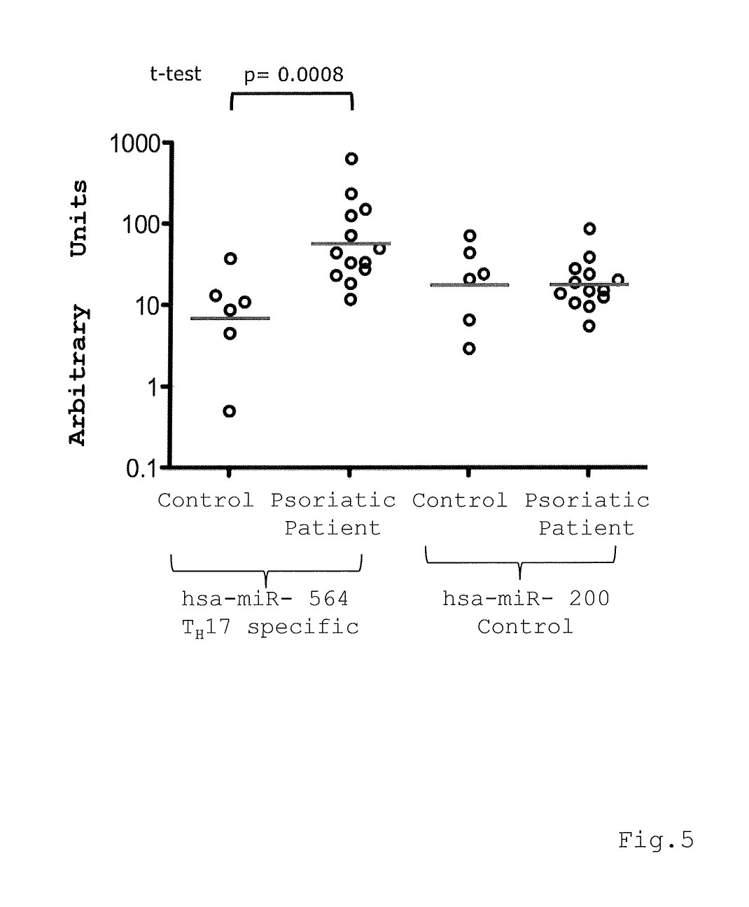

FIG. 5 shows the results of the quantitative RT-PCR analysis of the miRNAs hsa-miR-564 and hsa-miR-200 in the blood of patients affected by psoriatic arthritis compared to healthy donors.

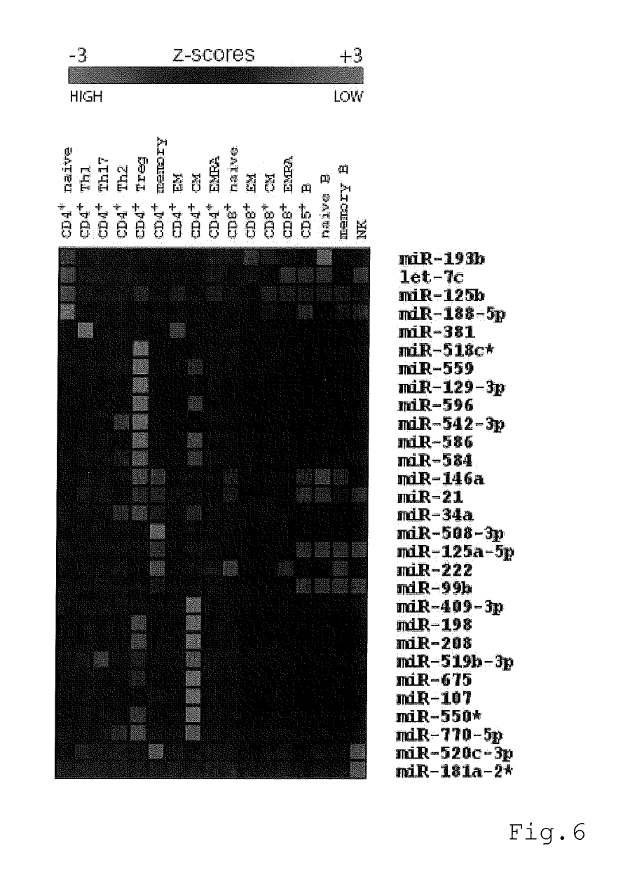

FIG. 6 shows a graphic representation by colour gradient (heatmap) of the characteristic miRNA expression profiles of human primary lymphocyte subpopulations; in particular, the miRNAs considered are those characterised by an expression which is 3 times higher than their expression evaluated in a some cell subpopulation.

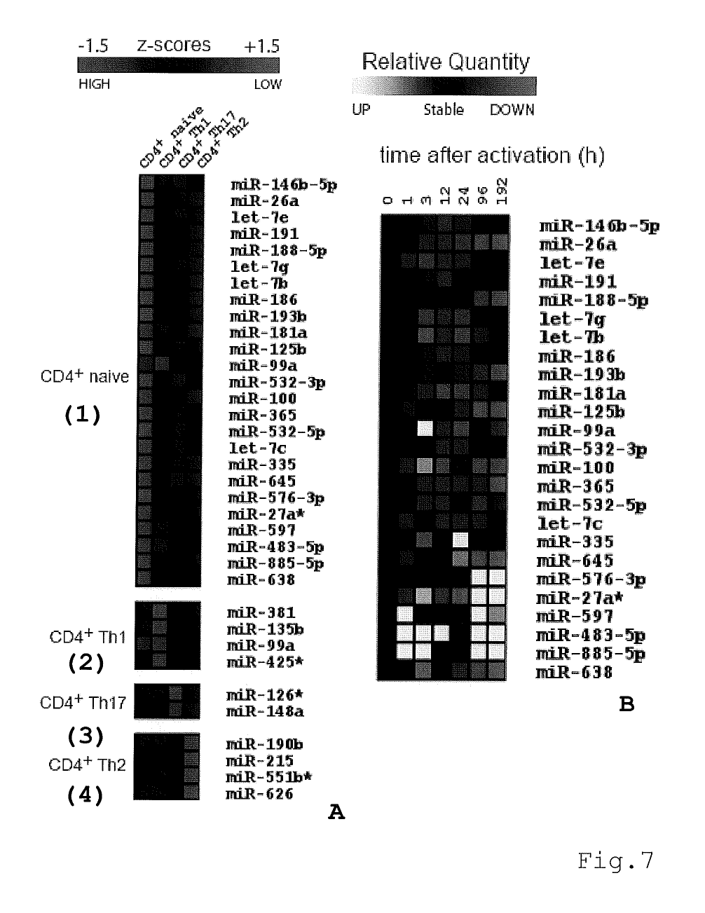

FIG. 7A shows a graphic representation by colour gradient (heatmap): (1) of the miRNA expression profiles specifically expressed in naive CD4⁺ T lymphocytes compared to T_{H}1, T_{H}2 and T_{H}17 lymphocytes; (2) of the miRNA expression profiles specifically expressed in T_{H}1 compared to naive CD4⁺ T, T_{H}2 and T_{H}17 lymphocytes; (3) of the miRNA expression profiles specifically expressed in T_{H}2 compared to naive CD4⁺ T, T_{H}1 and T_{H}17 lymphocytes; and (4) of the miRNA expression profiles specifically expressed in T_{H}17 compared to naive CD4⁺ T, T_{H}1 and T_{H}2 lymphocytes.

FIG. 7B shows the variation in the miRNA expression profiles specifically expressed in naive CD4⁺ T lymphocytes during their differentiation into T_{H}1, T_{H}2 and T_{H}17 memory lymphocyte cells (i.e. following activation of the naive CD4⁺ T lymphocytes).

According to the invention, a pathological condition caused by or associated with an immune system dysfunction means a condition in which the immune system shows improper functioning that may have the effect of compromising the body's integrity.

Preferably, said pathological condition caused by or associated with an immune system dysfunction is selected from among immunodeficiencies, neoplasia of the immune system and immune-mediated pathologies.

The immune-mediated pathologies are preferably, selected from among: an allergic condition and an autoimmune pathology. Said autoimmune pathology is preferably selected from among: systemic lupus erythematosus, rheumatoid arthritis, ankylosing spondylitis, multiple sclerosis, type 1 diabetes mellitus and psoriatic arthritis.

Preferably, the functionality of the immune system in an individual affected by a pathological condition according to the present invention or the response of an individual's immune system following a vaccination is assessed by monitoring the functioning of the lymphocyte populations, in particular T lymphocytes.

Preferably, such T lymphocytes are T helper lymphocytes expressing the protein CD4; more preferably they are naive CD4⁺ T, T_{H}1, T_{H}2, T_{H}17 lymphocytes or combinations thereof. Said monitoring is carried out, in particular, by measuring the expression level of at least one miRNA gene product expressed by said lymphocyte populations.

Monitoring said lymphocytes can be useful for diagnosing or prognosticating or evaluating the risk of developing an immune-mediated pathology or a pathological condition caused by or associated with a naive CD4⁺ T-dependent, T_{H}1-dependent, T_{H}2-dependent or T_{H}17-dependent dysfunction of the immune system, and for monitoring the effectiveness of a therapy against an immune-mediated pathology or a pathological condition caused by or associated with a naive CD4⁺ T-dependent, T_{H}1-dependent, T_{H}2-dependent or T_{H}17-dependent dysfunction of the immune system, using the method of the invention, which is based on comparing the expression levels of the gene product of specific miRNAs (in the blood or biological fluids of a patient) expressed by naive CD4⁺ T, T_{H}1, T_{H}2 or T_{H}17 lymphocytes before and after the onset of a naive CD4⁺ T-dependent, T_{H}1-dependent, T_{H}2-dependent or T_{H}17-dependent pathological condition, or at different stages of said pathological condition compared to a control level.

In particular, the allergic conditions can be caused by or associated with alterations in the normal functioning of naive CD4⁺ T and/or T_{H}2 lymphocytes. These cell populations can be used to diagnose or prognosticate or evaluate the risk of developing an allergy, or for monitoring the effectiveness of a therapy against an allergy, using the method of the invention, which is based on comparing the levels of specific miRNAs (in the blood or biological fluids of a patient) expressed by naive CD4⁺ T and/or T_{H}2 lymphocytes before and after the onset of the allergic condition, or at different stages of the allergic condition compared to a control level. In particular, the autoimmune pathologies, e.g. systemic lupus erythematosus, rheumatoid arthritis, ankylosing spondylitis, multiple sclerosis, type 1 diabetes mellitus and psoriatic arthritis, can be caused by or associated with alterations in the normal functioning of naive CD4⁺ T and/or T_{H}17 lymphocytes. These cell populations can be used to diagnose or prognosticate an autoimmune pathology, or evaluate the risk of developing an autoimmune pathology, or for monitoring the effectiveness of a therapy against an autoimmune pathology, using the method of the invention, which is based on comparing the levels of specific miRNAs (in the blood or biological fluids of a patient) expressed by naive CD4⁺ T and/or T_{H}17 lymphocytes before and after the onset of the autoimmune pathology, or at different stages of the autoimmune pathology compared to a control level.

Some states of the immune response are brought about by an induced physiological reaction of the naive CD4⁺ T lymphocytes and/or T_{H}1 lymphocytes, for example following a vaccination, in which an antigen administered in attenuated form evokes an immune response.

Naive CD4⁺ T and/or T_{H}1 lymphocytes can thus be used to monitor the follow-up of a vaccination, using the method of the invention, which is based on comparing the levels of specific miRNAs (in the blood or biological fluids of a patient) expressed by naive CD4⁺ T and/or T_{H}1 lymphocytes before and after the vaccination or at different stages of the response to a vaccination compared to a control level. miRNAs are molecules naturally present in many organisms, including animals, plants and viruses, and play a fundamental role in the control of gene expression by regulating, in a specific manner, the stability and translation of messenger RNAs (mRNAs). miRNAs are initially expressed as long precursor RNA molecules, or pri-miRNAs, which by means of a complex mechanism of nucleo-cytoplasmic processing, are transformed into the mature form (miRNA), characterised by a length of 17-24 nucleotides. The function of many miRNAs is not known; however, various studies have demonstrated the key role that miRNAs have in gene regulation in many fundamental biological functions such as apoptosis, haematopoietic development and cell differentiation.

The biological and clinical relevance of miRNAs expression profiles has been demonstrated in solid human tumours (like breast tumours) and chronic lymphatic leukaemia.

A further property of miRNAs is their presence, in a stable, RNA-resistant form, in blood (serum and plasma) and in various other biological fluids. It has recently been demonstrated that the blood of patients affected by prostate carcinoma or ovarian cancer shows peculiar miRNA expression profiles.

For the purposes of the present invention, the at least one miRNA gene product used in the method is at least one miRNA. The at least one miRNA gene product is chosen, individually or in combination, from the group consisting of SEQ ID NO: 1-154.

In a preferred embodiment of the invention, the at least one miRNA gene product is selected, individually or in combination, from the group consisting of SEQ ID NO: 1-3 and SEQ ID NO: 19-58, SEQ ID NO: 10, SEQ ID NO: 14, SEQ ID NO: 67-101, SEQ ID NO: 5, SEQ ID NO: 18, 102, 109, 111-138 and 154; more preferably it is selected from the group consisting of: SEQ ID NO: 1, 3, 18, 27, 32-33, 48, 58, 67, 79, 84, 92, 111-116, 118-119, 121-124, 126, 128, 130, 132-133, 137 and 154.

Said miRNA sequences are characterised by a higher relative expression level in a sample of a subject affected by a pathological condition caused by or associated with an immune system dysfunction compared to a control, or in a sample of a subject on whom a vaccination was performed compared to a control.

In another preferred embodiment of the present invention, the at least one miRNA gene product is selected, individually or in combination, from the group consisting of SEQ ID NO: 4-18 and SEQ ID NO: 59-66, SEQ ID NO: 102-110 and SEQ ID NO: 139-153 and SEQ ID NO: 37, 92; more preferably it is selected from the group consisting of: SEQ ID NO: 9, 18, 58, 105, 144, 149, 152 and 153.

Said miRNA sequences are characterised by a lower relative expression level in a sample of a subject affected by a pathological condition caused by or associated with an immune system dysfunction compared to a control, or in a sample of a subject on whom a vaccination was performed compared to a control.

A further embodiment of the present invention relates to the at least one miRNA gene product selected, individually or in combination, from the group consisting of SEQ ID NO: 18, 102, 109 and SEQ ID NO: 111-138, preferably, said group consists of SEQ ID NO: 18, 111-116, 118-119, 121-124, 126, 128, 130, 132-134 and 137.

Said miRNA sequences are characterized by a higher relative expression level in a sample of a subject affected by a pathological condition caused by or associated with an immune system dysfunction compared to a control, or in a sample of a subject on whom a vaccination was performed compared to a control.

In particular, said miRNAs are overexpressed by naive CD4⁺ T lymphocyte populations.

In a further embodiment of the present invention, the at least one miRNA gene product is selected, individually or in combination, from the group consisting of SEQ ID NO: 1-3, preferably said group consists of SEQ ID NO: 1 and SEQ ID NO: 3.

Said miRNA sequences are characterized by a higher relative expression level in a sample of a subject on whom a vaccination was performed compared to a control.

In particular, said miRNAs are overexpressed by T_{H}1 lymphocyte populations.

Another embodiment of the invention describes the at least one miRNA gene product selected, individually or in combination, from the group consisting of SEQ ID NO: 19-58, SEQ ID NO: 10, SEQ ID NO: 14 and SEQ ID NO: 154, preferably, said group consists of SEQ ID NO: 27, SEQ ID NO: 32, SEQ ID NO: 48 and SEQ ID NO: 154.

Said miRNA sequences are characterized by a higher relative expression level in a sample of a subject affected by an allergy compared to a control.

In particular, said miRNAs are overexpressed by T_{H}2 lymphocyte populations.

A further embodiment of the present invention relates to the at least one miRNA gene product selected, individually or in combination, from the group consisting of SEQ ID NO: 67-101 and SEQ ID NO: 5, preferably, the at least one miRNA gene product is SEQ ID NO: 67.

Said miRNA sequences are characterized by a higher relative expression level in a sample of a subject affected by an autoimmune disease compared to a control.

In particular, said miRNAs are overexpressed by T_{H}17 lymphocyte populations.

A further embodiment of the present invention relates to the at least one miRNA gene product selected, individually or in combination, from the group consisting of SEQ ID NO: 37, 92 and SEQ ID NO: 139-153, preferably, the at least one miRNA gene product is selected, individually or in combination, from the group consisting of: SEQ ID NO: 58, SEQ ID NO: 144, SEQ ID NO: 149 and SEQ ID NO: 152-153.

Said miRNA sequences are characterized by a lower relative expression level in a sample of a subject affected by a pathological condition caused by or associated with an immune system dysfunction compared to a control, or in a sample of a subject on whom a vaccination was performed compared to a control.

In particular, said miRNAs are underexpressed by naive CD4⁺ T lymphocyte populations.

In a further embodiment of the present invention, the at least one miRNA gene product is selected, individually or in combination, from the group consisting of SEQ ID NO: 4-18 preferably, said group consists of SEQ ID NO: 9 and SEQ ID NO: 18.

Said miRNA sequences are characterized by a lower relative expression level in a sample of a subject on whom a vaccination was performed compared to a control.

In particular, said miRNAs are underexpressed in T_{H}1 lymphocyte populations.

Another embodiment of the invention describes the at least one miRNA gene product selected, individually or in combination, from the group consisting of SEQ ID NO: 59-66.

Said miRNA sequences are characterized by a lower relative expression level in a sample of a subject affected by an allergy compared to a control.

In particular, said miRNAs are underexpressed by T_{H}2 lymphocyte populations.

A further embodiment of the present invention relates to the at least one miRNA gene product selected, individually or in combination, from the group consisting of SEQ ID NO: 102-110, preferably, said at least one miRNA gene product is SEQ ID NO: 105.

Said miRNA sequences are characterized by a lower relative expression level in a sample of a subject affected by an autoimmune disease compared to a control.

In particular, said miRNAs are underexpressed by T_{H}17 lymphocyte populations.

In a preferred embodiment of the present invention the at least one miRNA gene product is selected from among the sequences: SEQ ID NO: 18, 37, 92, 102, 109 and 111-153 and is overexpressed or underexpressed in a subject affected by a pathological condition caused by or associated with an immune system dysfunction compared to a control, or in a sample of a subject on whom a vaccination was performed compared to a control. More preferably, the at least one miRNA gene product is selected from among: SEQ ID NO: 18, 111-116, 118, 119, 121-124, 126, 128, 130, 132-134 and 137 and is overexpressed in a subject affected by a pathological condition caused by or associated with an immune system dysfunction compared to a control, or in a sample of a subject on whom a vaccination was performed compared to a control; and/or the at least one miRNA gene product is selected from among: SEQ ID NO: 58, 144, 149 and 152-153 and is underexpressed in a subject affected by a pathological condition caused by or associated with an immune system dysfunction compared to a control, or in a sample of a subject on whom a vaccination was performed compared to a control. Preferably, the at least one miRNA gene product selected from among: SEQ ID NO: 18, 111-116, 118, 119, 121-124, 126, 128, 130, 132-134 and 137 and/or the at least one miRNA gene product selected from among: SEQ ID NO: 58, 144, 149 and 152-153 is overexpressed and/or underexpressed by the naive CD4⁺ T lymphocytes of said subject.

In a preferred embodiment of the present invention the at least one miRNA gene product is selected from among the sequences: SEQ ID NO: 1-18, preferably SEQ ID NO: 1, 3, 9 and 18, and is overexpressed or underexpressed in a subject on whom a vaccination was performed compared to a control. More preferably, the at least one miRNA gene product is selected from among: SEQ ID NO: 1 and 3 and is overexpressed in a subject on whom a vaccination was performed compared to a control; and/or the at least one miRNA gene product is selected from among the sequences SEQ ID NO: 9 and 18 and is underexpressed in a subject on whom a vaccination was performed compared to a control. Preferably, the at least one miRNA gene product selected from among: SEQ ID NO: 1 and 3 and/or the at least one miRNA gene product selected from among the sequences SEQ ID NO: 9 and 18 is overexpressed and/or underexpressed by the T_{H}1 lymphocytes of said subject. In a preferred embodiment of the present invention the at least one miRNA gene product is selected from among the sequences: SEQ ID NO: 19-66, SEQ ID NO: 10, SEQ ID NO: 14 and SEQ ID NO: 154, preferably SEQ ID NO: 27, 32, 33, 48, 58 and 154, and is overexpressed or underexpressed in a subject affected by an allergy compared to a control. More preferably, the at least one miRNA gene product is selected from among: SEQ ID NO:27, SEQ ID NO: 32, SEQ ID NO: 48 and SEQ ID NO: 154 and is overexpressed in a subject affected by an allergy compared to a control; preferably, it is overexpressed by the T_{H}2 lymphocytes of said subject.

In a preferred embodiment of the present invention the at least one miRNA gene product is selected from among the sequences: SEQ ID NO: 67-110 and SEQ ID NO: 5, preferably SEQ ID NO: 67, 79, 84, 92 and 105 and is overexpressed or underexpressed in a subject affected by an autoimmune disease compared to a control. More preferably, the at least one miRNA gene product is SEQ ID NO: 67 and is overexpressed in a subject affected by an autoimmune disease compared to a control; and/or the at least one miRNA gene product is SEQ ID NO: 105 and is underexpressed in a subject affected by an autoimmune disease compared to a control. Preferably, SEQ ID NO: 67 and/or SEQ ID NO: 105 are overexpressed and/or underexpressed by the T_{H}17 lymphocytes of said subject.

The method of the present invention is preferably carried out in vitro, in particular on blood or biological fluid samples of a human subject.

The peripheral blood sample to be investigated can be whole blood, peripheral blood mononuclear cells, serum or plasma isolated (ex vivo).

The sample to be investigated can also be any biological fluid, for example urine or saliva.

The method described relates to a pathological condition caused by or associated with an immune system dysfunction of an individual, in particular, said condition is an allergy or an autoimmune disease, in an advanced or even early stage.

The method of the invention is used to diagnose whether a subject is affected by a pathological condition caused by or associated with an immune system dysfunction, or whether there is a risk of developing such a pathological condition, by checking for an alteration in the expression levels of at least one miRNA gene product in a peripheral blood or biological fluid sample of the test subject, compared to a control sample or level.

The method of the invention is also used to define the prognosis of a pathological condition caused by or associated with an immune system dysfunction by comparing the expression levels of at least one miRNA gene product in a peripheral blood or biological fluid sample of a subject affected by a pathological condition caused by or associated with an immune system dysfunction with a reference level. An alteration in the expression levels of the at least one miRNA gene product in a sample of the test subject, compared to a reference sample, is indicative of the degree of advancement of the pathological condition, from which it is possible to deduce a prognosis of the condition itself.

The method of the invention is also used to monitor the effectiveness of a therapeutic treatment targeted against a pathological condition caused by or associated with an immune system dysfunction, in particular the treatment of an allergy or autoimmune pathology, in particular systemic lupus erythematosus, rheumatoid arthritis, ankylosing spondylitis, multiple sclerosis, type 1 diabetes mellitus and psoriatic arthritis.

In this case the method comprises comparing the expression levels of at least one miRNA gene product in a peripheral blood or biological fluid sample of the test subject with a reference sample or level. An alteration in the expression levels of the at least one miRNA gene product in a sample of the test subject, compared to a sample of the same subject in different phases of the therapeutic treatment in question, is indicative of the effectiveness of the treatment itself. Alternatively, the method for determining the effectiveness of a therapeutic treatment targeted against a pathological condition caused by or associated with an immune system dysfunction comprises comparing the expression levels of at least one miRNA gene product in a sample of peripheral blood of a patient affected by a pathological condition caused by or associated with an immune system dysfunction and who is undergoing a therapeutic treatment targeted against said pathological condition, with a sample of peripheral blood of a patient affected by a pathological condition caused by or associated with an immune system dysfunction and who is not undergoing a therapeutic treatment targeted against said pathological condition. A difference in the expression levels of the miRNA gene product between the two groups of patients is indicative of whether a new method of therapeutic treatment targeted against a pathological condition caused by or associated with an immune system dysfunction is effective or not.

The method of the invention is also used for the follow up of a vaccination. In this case the method comprises comparing the expression levels of at least one miRNA gene product in a peripheral blood sample from the vaccinated subject compared to a control, in the days following administration of the vaccine and any booster shots, preferably 15-30 days after the vaccination.

In another embodiment, the method according to the present invention can also be used in combination with other diagnostic/prognostic methods presently in use, as a valid complement to said investigative techniques.

For example, the method can be applied in combination with: microarray, proteomic and immunological analysis, and sequencing analysis of specific DNA sequences for the purpose of defining an ad hoc therapeutic approach for individual patients. Completing the clinical information derived from known investigative techniques with that of the present invention would help to address the treatment of a patient affected by a pathological condition caused by or associated with an immune system dysfunction in a completely personalised manner that is advantageous as regards both the risk of developing a pathological condition caused by or associated with an immune system dysfunction, and for the diagnosis and prognosis of and therapy for a pathological condition caused by or associated with an immune system dysfunction.

In another embodiment, the method of the invention can be used to identify new therapeutic targets.

In fact, each miRNA has the capability of regulating the expression of hundreds of genes and can thus modulate the activity of many molecular signal transduction pathways inside the cell. Therefore, the miRNA panels identified in the peripheral blood of a subject affected by a pathological condition caused by or associated with an immune system dysfunction reflect the biology of the damage or primary tumour.

Said miRNAs are useful as biomarkers for identifying the pathological condition, defining the response to therapies and monitoring any possible recurrences of a pathological condition caused by or associated with an immune system dysfunction. Said miRNAs are also useful for defining the altered molecular pathways in a pathological condition caused by or associated with an immune system dysfunction and can thus contribute to identifying new therapeutic targets.

The present invention also relates to a pharmaceutical composition for treating a pathological condition caused by or associated with an immune system dysfunction, comprising a pharmaceutically acceptable carrier and at least one isolated miRNA gene product and/or a nucleic acid complementary thereto, which is up- or down-regulated in the peripheral blood of a subject affected by a pathological condition caused by or associated with an immune system dysfunction, compared to a suitable control sample. The at least one isolated miRNA gene product is selected, individually or in different combinations, from among the sequences previously identified.

The present invention further relates to a method for identifying a pathological condition caused by or associated with an immune system dysfunction which comprises a step of administering a test substance to (ex vivo) isolated cells. After administration, a measurement is made of the level of at least one miRNA gene product whose increased expression is associated with a pathological condition caused by or associated with an immune system dysfunction.

Subsequently, the expression level of said at least one miRNA gene product in the treated cells is compared with that in the control cells. A decrease in said expression level is indicative of the fact that the test substance is useful in treating the pathological condition caused by or associated with an immune system dysfunction.

### EXPERIMENTAL PART

### Example 1

The analysis were carried out on T_{H}1, T_{H}2, T_{H}17 and naive CD4⁺ T lymphocytes isolated from peripheral blood of healthy donors. The total RNA was extracted using the mirVana™ miRNA Isolation Kit (Cat# AM1561-Ambion). An aliquot of the extracted sample (10 ng of total RNA) was submitted to a reverse transcription reaction conducted using the TaqMan® MicroRNA Reverse Transcription kit in the presence of a solution of MgCl₂ 5 mM (Part no. 4366597—Applied Biosystems). Megaplex™ RT Primers were used as primers for the reverse transcription, a set of 2 predefined pools (Pool A and Pool B) of 380 RT primers each, which permits the simultaneous synthesis of cDNAs from mature miRNAs (Megaplex™ RT Primers Human Pool A, Part No.: 4399966; Human Pool B, Part No.: 4399968—Applied Biosystems). Final reaction volume (μL): 7.5.

Incubation Conditions for a Reaction Cycle:

16° C. 2 min

42° C. 1 min

50° C. 1 sec

85° C. 5 min

4° C. ∞

(for 40 cycles)

The cDNA thus produced was pre-amplified (2.5 μL of the 7.5) using TaqMan PreAmp Master Mix (2×) (Part No.: 4384266—Applied Biosystems) and Megaplex™ PreAmp Primers, a set of 2 pools of gene-specific, forward and reverse primers (Megaplex™ PreAmp Primers, Human Pool A, Part no. 4399233; Human Pool B Part no. 4399201—Applied Biosystems). Final reaction volume (μL): 25.

Incubation Conditions:

95° C. 10 min

55° C. 2 min

72° C. 2 min

95° C. 15 sec

60° C. 4 min×12 cycles

4° C. ∞

The pre-amplified cDNA was used for the real-time PCR reaction. The reaction was conducted using TaqMan Universal PCR Master Mix, No Amperase UNG, 2×(Part No: 4326614—Applied Biosystems) in 900 final μL, loaded onto 2 sets of microfluidic cards, TaqMan® Human MicroRNA Low Density Arrays (Part No.: 4400238— Applied Biosystems), with 384 wells each, containing TaqMan probes. Said analysis (Array A and Array B) enables quantification of the gene expression levels of 665 miRNAs and of the respective controls (http://www3.appliedbiosystems.com/cms/groups/portal/documents/generaldocuments/cms052133.xls).

The average Ct value of three different cell snRNAs, U6 snRNA, RNU44 and RNU48, can be used as an internal control for calculating the relative gene expression. The relative expression of each miRNA can be calculated using the equation 2^{-ΔCt}, where ΔCt=(Ct miRNA)−(internal ctrl Ct).

The relative expression of each miRNA as determined by means of PCR can be calculated using standard methods whereby the lymphocyte populations considered are taken in turn as a reference and compared with the other remaining lymphocyte populations. The expression data (ΔCt) obtained for each miRNA are compared, and the miRNAs selected are the ones for which there is a difference greater than 1.5 (in absolute value) between the ΔCt value of the miRNA in the reference population and the corresponding ΔCt in all the other populations.

FIGS. 1, 2, 3 and 4 show the results of these selections displayed in “heatmap” graphics for the four lymphocyte populations, in a white/black expression gradient (white for expressed and black for unexpressed) and in two groups per population: the ones which were overexpressed in the reference population (top panel) and the ones which were underexpressed in the reference population (bottom panel). RT-PCR quantitative analysis showed the presence of 18 miRNAs, listed in Table 1, which are present in higher or lower quantity in the T_{H}1 lymphocytes than in the T_{H}2 or T_{H}17 lymphocytes or naive CD4⁺ T cells.

TABLE 1

| | | miRNA sequence |
|---|---|---|
| miRNA name | miRNA sequence | No. |
| hsa-miR-135b | UAUGGCUUUUCAUUCCUAUGUGA | SEQ ID NO: 1 |
| hsa-miR-375 | UUUGUUCGUUCGGCUCGCGUGA | SEQ ID NO: 2 |
| hsa-miR-381 | UAUACAAGGGCAAGCUCUCUGU | SEQ ID NO: 3 |
| hsa-miR-128 | UCACAGUGAACCGGUCUCUUU | SEQ ID NO: 4 |
| hsa-miR-199a-3p | ACAGUAGUCUGCACAUUGGUUA | SEQ ID NO: 5 |
| hsa-miR-200b | UAAUACUGCCUGGUAAUGAUGA | SEQ ID NO: 6 |
| hsa-miR-339-5p | UCCCUGUCCUCCAGGAGCUCACG | SEQ ID NO: 7 |
| hsa-miR-423-5p | UGAGGGGCAGAGAGCGAGACUUU | SEQ ID NO: 8 |
| hsa-miR-425* | AUCGGGAAUGUCGUGUCCGCCC | SEQ ID NO: 9 |
| hsa-miR-489 | GUGACAUCACAUAUACGGCAGC | SEQ ID NO: 10 |
| hsa-miR-505* | GGGAGCCAGGAAGUAUUGAUGU | SEQ ID NO: 11 |
| hsa-miR-513-3p | UAAAUUUCACCUUUCUGAGAAGG | SEQ ID NO: 12 |
| hsa-miR-516a-3p | UGCUUCCUUUCAGAGGGU | SEQ ID NO: 13 |
| hsa-miR-520d-5p | CUACAAAGGGAAGCCCUUUC | SEQ ID NO: 14 |
| hsa-miR-523 | GAACGCGCUUCCCUAUAGAGGGU | SEQ ID NO: 15 |
| hsa-miR-643 | ACUUGUAUGCUAGCUCAGGUAG | SEQ ID NO: 16 |
| hsa-miR-801 | GAUUGCUCUGCGUGCGGAAUCGAC | SEQ ID NO: 17 |
| hsa-miR-99a | AACCCGUAGAUCCGAUCUUGUG | SEQ ID NO: 18 |

In particular, Table 2 shows the miRNAs present in a higher quantity in the T_{H}1 lymphocytes than in the T_{H}2 or T_{H}17 lymphocytes or naive CD4⁺ T cells.

TABLE 2
List of the miRNAs overexpressed in the T_{H}1 lymphocytes
(compared to naive and other T helpers), with a minimum of
1.5 CT

| miRNA name | miRNA sequence | Sequence Number |
|---|---|---|
| hsa-miR-135b | UAUGGCUUUUCAUUCCUAUGUGA | SEQ ID NO: 1 |
| hsa-miR-375 | UUUGUUCGUUCGGCUCGCGUGA | SEQ ID NO: 2 |
| hsa-miR-381 | UAUACAAGGGCAAGCUCUCUGU | SEQ ID NO: 3 |

In particular, Table 3 shows the miRNAs present in a lower quantity in the T_{H}1 lymphocytes than in the T_{H}2 or T_{H}17 lymphocytes or naive CD4⁺ T cells.

TABLE 3
List of the miRNAs underexpressed in the T_{H}1 lymphocytes
(compared to naive and other T helpers), with a minimum of
1.5 CT

| miRNA name | miRNA sequence | Sequence Number |
|---|---|---|
| hsa-miR-128 | UCACAGUGAACCGGUCUCUUU | SEQ ID NO: 4 |
| hsa-miR-199a-3p | ACAGUAGUCUGCACAUUGGUUA | SEQ ID NO: 5 |
| hsa-miR-200b | UAAUACUGCCUGGUAAUGAUGA | SEQ ID NO: 6 |
| hsa-miR-339-5p | UCCCUGUCCUCCAGGAGCUCACG | SEQ ID NO: 7 |
| hsa-miR-423-5p | UGAGGGGCAGAGAGCGAGACUUU | SEQ ID NO: 8 |
| hsa-miR-425* | AUCGGGAAUGUCGUGUCCGCCC | SEQ ID NO: 9 |
| hsa-miR-489 | GUGACAUCACAUAUACGGCAGC | SEQ ID NO: 10 |
| hsa-miR-505* | GGGAGCCAGGAAGUAUUGAUGU | SEQ ID NO: 11 |
| hsa-miR-513-3p | UAAAUUUCACCUUUCUGAGAAGG | SEQ ID NO: 12 |
| hsa-miR-516a-3p | UGCUUCCUUUCAGAGGGU | SEQ ID NO: 13 |
| hsa-miR-520d-5p | CUACAAAGGGAAGCCCUUUC | SEQ ID NO: 14 |
| hsa-miR-523 | GAACGCGCUUCCCUAUAGAGGGU | SEQ ID NO: 15 |
| hsa-miR-643 | ACUUGUAUGCUAGCUCAGGUAG | SEQ ID NO: 16 |
| hsa-miR-801 | GAUUGCUCUGCGUGCGGAAUCGAC | SEQ ID NO: 17 |
| hsa-miR-99a | AACCCGUAGAUCCGAUCUUGUG | SEQ ID NO: 18 |

### Example 2

The analysis were carried out on T_{H}2 lymphocytes isolated from peripheral blood of healthy donors. RT-PCR quantitative analysis, conducted as in example 1, showed the presence of 50 miRNAs, described in Table 4, which are present in higher or lower quantity in the T_{H}2 lymphocytes than in the T_{H}1 or T_{H}17 lymphocytes or naive CD4⁺ T cells.

TABLE 4

| miRNA name | miRNA sequence | Sequence Number |
|---|---|---|
| hsa-let-7g* | CUGUACAGGCCACUGCCUUGC | SEQ ID NO: 19 |
| hsa-miR-1 | UGGAAUGUAAAGAAGUAUGUAU | SEQ ID NO: 20 |
| hsa-miR-127-5p | CUGAAGCUCAGAGGGCUCUGAU | SEQ ID NO: 21 |
| hsa-miR-132* | ACCGUGGCUUUCGAUUGUUACU | SEQ ID NO: 22 |
| hsa-miR-136 | ACUCCAUUUGUUUUGAUGAUGGA | SEQ ID NO: 23 |
| hsa-miR-136* | CAUCAUCGUCUCAAAUGAGUCU | SEQ ID NO: 24 |
| hsa-miR-145 | GUCCAGUUUUCCCAGGAAUCCCU | SEQ ID NO: 25 |
| hsa-miR-18b | UAAGGUGCAUCUAGUGCAGUUAG | SEQ ID NO: 26 |
| hsa-miR-190b | UGAUAUGUUUGAUAUUGGGUU | SEQ ID NO: 27 |
| hsa-miR-198 | GGUCCAGAGGGGAGAUAGGUUC | SEQ ID NO: 28 |
| hsa-miR-19a* | AGUUUUGCAUAGUUGCACUACA | SEQ ID NO: 29 |
| hsa-miR-208b | AUAAGACGAACAAAAGGUUUGU | SEQ ID NO: 30 |
| hsa-miR-210 | CUGUGCGUGUGACAGCGGCUGA | SEQ ID NO: 31 |
| hsa-miR-215 | AUGACCUAUGAAUUGACAGAC | SEQ ID NO: 32 |
| hsa-miR-22* | AGUUCUUCAGUGGCAAGCUUUA | SEQ ID NO: 33 |
| hsa-miR-24-1* | UGCCUACUGAGCUGAUAUCAGU | SEQ ID NO: 34 |
| hsa-miR-302d | UAAGUGCUUCCAUGUUUGAGUGU | SEQ ID NO: 35 |
| hsa-miR-335* | UUUUUCAUUAUUGCUCCUGACC | SEQ ID NO: 36 |
| hsa-miR-34a | UGGCAGUGUCUUAGCUGGUUGU | SEQ ID NO: 37 |
| hsa-miR-378* | CUCCUGACUCCAGGUCCUGUGU | SEQ ID NO: 38 |
| hsa-miR-382 | GAAGUUGUUCGUGGUGGAUUCG | SEQ ID NO: 39 |
| hsa-miR-449b | AGGCAGUGUAUUGUUAGCUGGC | SEQ ID NO: 40 |
| hsa-miR-486-5p | UCCUGUACUGAGCUGCCCCGAG | SEQ ID NO: 41 |
| hsa-miR-489 | GUGACAUCACAUAUACGGCAGC | SEQ ID NO: 10 |
| hsa-miR-496 | UGAGUAUUACAUGGCCAAUCUC | SEQ ID NO: 42 |
| hsa-miR-501-5p | AAUCCUUUGUCCCUGGGUGAGA | SEQ ID NO: 43 |
| hsa-miR-518b | CAAAGCGCUCCCCUUUAGAGGU | SEQ ID NO: 44 |
| hsa-miR-518d-3p | CAAAGCGCUUCCCUUUGGAGC | SEQ ID NO: 45 |
| hsa-miR-518e | AAAGCGCUUCCCUUCAGAGUG | SEQ ID NO: 46 |
| hsa-miR-520d-5p | CUACAAAGGGAAGCCCUUUC | SEQ ID NO: 14 |
| hsa-miR-542-3p | UGUGACAGAUUGAUAACUGAAA | SEQ ID NO: 47 |
| hsa-miR-551b* | GAAAUCAAGCGUGGGUGAGACC | SEQ ID NO: 48 |
| hsa-miR-567 | AGUAUGUUCUUCCAGGACAGAAC | SEQ ID NO: 49 |
| hsa-miR-583 | CAAAGAGGAAGGUCCCAUUAC | SEQ ID NO: 50 |
| hsa-miR-589* | UCAGAACAAAUGCCGGUUCCCAGA | SEQ ID NO: 51 |
| hsa-miR-603 | CACACACUGCAAUUACUUUUGC | SEQ ID NO: 52 |
| hsa-miR-605 | UAAAUCCCAUGGUGCCUUCUCCU | SEQ ID NO: 53 |
| hsa-miR-609 | AGGGUGUUUCUCUCAUCUCU | SEQ ID NO: 54 |
| hsa-miR-615-3p | UCCGAGCCUGGGUCUCCCUCUU | SEQ ID NO: 55 |
| hsa-miR-639 | AUCGCUGCGGUUGCGAGCGCUGU | SEQ ID NO: 56 |
| hsa-miR-675 | UGGUGCGGAGAGGGCCCACAGUG | SEQ ID NO: 57 |
| hsa-miR-885-5p | UCCAUUACACUACCCUGCCUCU | SEQ ID NO: 58 |
| hsa-miR-130b | CAGUGCAAUGAUGAAAGGGCAU | SEQ ID NO: 59 |
| hsa-miR-27b | UUCACAGUGGCUAAGUUCUGC | SEQ ID NO: 60 |
| hsa-miR-32 | UAUUGCACAUUACUAAGUUGCA | SEQ ID NO: 61 |
| hsa-miR-323-3p | CACAUUACACGGUCGACCUCU | SEQ ID NO: 62 |
| hsa-miR-339-3p | UGAGCGCCUCGACGACAGAGCCG | SEQ ID NO: 63 |
| hsa-miR-362-5p | AAUCCUUGGAACCUAGGUGUGAGU | SEQ ID NO: 64 |
| hsa-miR-425 | AAUGACACGAUCACUCCCGUUGA | SEQ ID NO: 65 |
| hsa-miR-502-3p | AAUGCACCUGGGCAAGGAUUCA | SEQ ID NO: 66 |

In particular, the miRNAs shown in Table 5 are present in higher quantity in the T_{H}2 lymphocytes than in the T_{H}1 or T_{H}17 lymphocytes or naive CD4⁺ T cells.

TABLE 5
List of the miRNAs overexpressed in the T_{H}2 lymphocytes
(compared to naive and other T helpers), with a minimum of
1.5 CT

| miRNA name | miRNA sequence | Sequence Number |
|---|---|---|
| hsa-let-7g* | CUGUACAGGCCACUGCCUUGC | SEQ ID NO: 19 |
| hsa-miR-1 | UGGAAUGUAAAGAAGUAUGUAU | SEQ ID NO: 20 |
| hsa-miR-127-5p | CUGAAGCUCAGAGGGCUCUGAU | SEQ ID NO: 21 |
| hsa-miR-132* | ACCGUGGCUUUCGAUUGUUACU | SEQ ID NO: 22 |
| hsa-miR-136 | ACUCCAUUUGUUUUGAUGAUGGA | SEQ ID NO: 23 |
| hsa-miR-136* | CAUCAUCGUCUCAAAUGAGUCU | SEQ ID NO: 24 |
| hsa-miR-145 | GUCCAGUUUUCCCAGGAAUCCCU | SEQ ID NO: 25 |
| hsa-miR-18b | UAAGGUGCAUCUAGUGCAGUUAG | SEQ ID NO: 26 |
| hsa-miR-190b | UGAUAUGUUUGAUAUUGGGUU | SEQ ID NO: 27 |
| hsa-miR-198 | GGUCCAGAGGGGAGAUAGGUUC | SEQ ID NO: 28 |
| hsa-miR-19a* | AGUUUUGCAUAGUUGCACUACA | SEQ ID NO: 29 |
| hsa-miR-208b | AUAAGACGAACAAAAGGUUUGU | SEQ ID NO: 30 |
| hsa-miR-210 | CUGUGCGUGUGACAGCGGCUGA | SEQ ID NO: 31 |
| hsa-miR-215 | AUGACCUAUGAAUUGACAGAC | SEQ ID NO: 32 |
| hsa-miR-22* | AGUUCUUCAGUGGCAAGCUUUA | SEQ ID NO: 33 |
| hsa-miR-24-1* | UGCCUACUGAGCUGAUAUCAGU | SEQ ID NO: 34 |
| hsa-miR-302d | UAAGUGCUUCCAUGUUUGAGUGU | SEQ ID NO: 35 |
| hsa-miR-335* | UUUUUCAUUAUUGCUCCUGACC | SEQ ID NO: 36 |
| hsa-miR-34a | UGGCAGUGUCUUAGCUGGUUGU | SEQ ID NO: 37 |
| hsa-miR-378* | CUCCUGACUCCAGGUCCUGUGU | SEQ ID NO: 38 |
| hsa-miR-382 | GAAGUUGUUCGUGGUGGAUUCG | SEQ ID NO: 39 |
| hsa-miR-449b | AGGCAGUGUAUUGUUAGCUGGC | SEQ ID NO: 40 |
| hsa-miR-486-5p | UCCUGUACUGAGCUGCCCCGAG | SEQ ID NO: 41 |
| hsa-miR-489 | GUGACAUCACAUAUACGGCAGC | SEQ ID NO: 10 |
| hsa-miR-496 | UGAGUAUUACAUGGCCAAUCUC | SEQ ID NO: 42 |
| hsa-miR-501-5p | AAUCCUUUGUCCCUGGGUGAGA | SEQ ID NO: 43 |
| hsa-miR-518b | CAAAGCGCUCCCCUUUAGAGGU | SEQ ID NO: 44 |
| hsa-miR-518d-3p | CAAAGCGCUUCCCUUUGGAGC | SEQ ID NO: 45 |
| hsa-miR-518e | AAAGCGCUUCCCUUCAGAGUG | SEQ ID NO: 46 |
| hsa-miR-520d-5p | CUACAAAGGGAAGCCCUUUC | SEQ ID NO: 14 |
| hsa-miR-542-3p | UGUGACAGAUUGAUAACUGAAA | SEQ ID NO: 47 |
| hsa-miR-551b* | GAAAUCAAGCGUGGGUGAGACC | SEQ ID NO: 48 |
| hsa-miR-567 | AGUAUGUUCUUCCAGGACAGAAC | SEQ ID NO: 49 |
| hsa-miR-583 | CAAAGAGGAAGGUCCCAUUAC | SEQ ID NO: 50 |
| hsa-miR-589* | UCAGAACAAAUGCCGGUUCCCAGA | SEQ ID NO: 51 |
| hsa-miR-603 | CACACACUGCAAUUACUUUUGC | SEQ ID NO: 52 |
| hsa-miR-605 | UAAAUCCCAUGGUGCCUUCUCCU | SEQ ID NO: 53 |
| hsa-miR-609 | AGGGUGUUUCUCUCAUCUCU | SEQ ID NO: 54 |
| hsa-miR-615-3p | UCCGAGCCUGGGUCUCCCUCUU | SEQ ID NO: 55 |
| hsa-miR-639 | AUCGCUGCGGUUGCGAGCGCUGU | SEQ ID NO: 56 |
| hsa-miR-675 | UGGUGCGGAGAGGGCCCACAGUG | SEQ ID NO: 57 |
| hsa-miR-885-5p | UCCAUUACACUACCCUGCCUCU | SEQ ID NO: 58 |

In particular, the miRNAs shown in Table 6 are present in lower quantity in the T_{H}2 lymphocytes than in the T_{H}1 or T_{H}17 lymphocytes or naive CD4⁺ T cells.

TABLE 6
List of the miRNAs underexpressed in the T_{H}2 lymphocytes
(compared to naive and other T helpers), with a minimum of
1.5 CT

| miRNA name | miRNA sequence | Sequence Number |
|---|---|---|
| hsa-miR-130b | CAGUGCAAUGAUGAAAGGGCAU | SEQ ID NO: 59 |
| hsa-miR-27b | UUCACAGUGGCUAAGUUCUGC | SEQ ID NO: 60 |
| hsa-miR-32 | UAUUGCACAUUACUAAGUUGCA | SEQ ID NO: 61 |
| hsa-miR-323-3p | CACAUUACACGGUCGACCUCU | SEQ ID NO: 62 |
| hsa-miR-339-3p | UGAGCGCCUCGACGACAGAGCCG | SEQ ID NO: 63 |
| hsa-miR-362-5p | AAUCCUUGGAACCUAGGUGUGAGU | SEQ ID NO: 64 |
| hsa-miR-425 | AAUGACACGAUCACUCCCGUUGA | SEQ ID NO: 65 |
| hsa-miR-502-3p | AAUGCACCUGGGCAAGGAUUCA | SEQ ID NO: 66 |

### Example 3

The analysis were carried out on T_{H}17 lymphocytes isolated from peripheral blood of healthy donors. Quantitative RT-PCR analysis, conducted as in example 1, showed the presence of 45 miRNAs, described in Table 7, which are present in higher or lower quantity in the T_{H}17 lymphocytes than in the T_{H}1 or T_{H}2 lymphocytes or naive CD4⁺ T cells.

TABLE 7

| | | Sequence |
|---|---|---|
| miRNA | Target Sequence | Identifier |
| hsa-miR-126* | CAUUAUUACUUUUGGUACGCG | SEQ ID NO: 67 |
| hsa-miR-130a | CAGUGCAAUGUUAAAAGGGCAU | SEQ ID NO: 68 |
| hsa-miR-143* | GGUGCAGUGCUGCAUCUCUGGU | SEQ ID NO: 69 |
| hsa-miR-144* | GGAUAUCAUCAUAUACUGUAAG | SEQ ID NO: 70 |
| hsa-miR-145* | GGAUUCCUGGAAAUACUGUUCU | SEQ ID NO: 71 |
| hsa-miR-181a* | ACCAUCGACCGUUGAUUGUACC | SEQ ID NO: 72 |
| hsa-miR-188-3p | CUCCCACAUGCAGGGUUUGCA | SEQ ID NO: 73 |
| hsa-miR-193a-3p | AACUGGCCUACAAAGUCCCAGU | SEQ ID NO: 74 |
| hsa-miR-199a-3p | ACAGUAGUCUGCACAUUGGUUA | SEQ ID NO: 5 |
| hsa-miR-19b-2* | AGUUUUGCAGGUUUGCAUUUCA | SEQ ID NO: 75 |
| hsa-miR-202* | UUCCUAUGCAUAUACUUCUUUG | SEQ ID NO: 76 |
| hsa-miR-208 | AUAAGACGAGCAAAAAGCUUGU | SEQ ID NO: 77 |
| hsa-miR-220 | CCACACCGUAUCUGACACUUU | SEQ ID NO: 78 |
| hsa-miR-221 | AGCUACAUUGUCUGCUGGGUUUC | SEQ ID NO: 79 |
| hsa-miR-29b-1* | GCUGGUUUCAUAUGGUGGUUUAGA | SEQ ID NO: 80 |
| hsa-miR-29c* | UGACCGAUUUCUCCUGGUGUUC | SEQ ID NO: 81 |
| hsa-miR-302a | UAAGUGCUUCCAUGUUUUGGUGA | SEQ ID NO: 82 |
| hsa-miR-324-5p | CGCAUCCCCUAGGGCAUUGGUGU | SEQ ID NO: 83 |
| hsa-miR-331-5p | CUAGGUAUGGUCCCAGGGAUCC | SEQ ID NO: 84 |
| hsa-miR-33a* | CAAUGUUUCCACAGUGCAUCAC | SEQ ID NO: 85 |
| hsa-miR-376a | AUCAUAGAGGAAAAUCCACGU | SEQ ID NO: 86 |
| hsa-miR-450b-5p | UUUUGCAAUAUGUUCCUGAAUA | SEQ ID NO: 87 |
| hsa-miR-519e | AAGUGCCUCCUUUUAGAGUGUU | SEQ ID NO: 88 |
| hsa-miR-524-3p | GAAGGCGCUUCCCUUUGGAGU | SEQ ID NO: 89 |
| hsa-miR-548d-5p | AAAAGUAAUUGUGGUUUUUGCC | SEQ ID NO: 90 |
| hsa-miR-550* | UGUCUUACUCCCUCAGGCACAU | SEQ ID NO: 91 |
| hsa-miR-564 | AGGCACGGUGUCAGCAGGC | SEQ ID NO: 92 |
| hsa-miR-566 | GGGCGCCUGUGAUCCCAAC | SEQ ID NO: 93 |
| hsa-miR-582-5p | UUACAGUUGUUCAACCAGUUACU | SEQ ID NO: 94 |
| hsa-miR-587 | UUUCCAUAGGUGAUGAGUCAC | SEQ ID NO: 95 |
| hsa-miR-623 | AUCCCUUGCAGGGGCUGUUGGGU | SEQ ID NO: 96 |
| hsa-miR-628-3p | UCUAGUAAGAGUGGCAGUCGA | SEQ ID NO: 97 |
| hsa-miR-649 | AAACCUGUGUUGUUCAAGAGUC | SEQ ID NO: 98 |
| hsa-miR-672 | UGAGGUUGGUGUACUGUGUGUGA | SEQ ID NO: 99 |
| hsa-miR-708 | AAGGAGCUUACAAUCUAGCUGGG | SEQ ID NO: 100 |
| hsa-miR-98 | UGAGGUAGUAAGUUGUAUUGUU | SEQ ID NO: 101 |
| hsa-let-7a | UGAGGUAGUAGGUUGUAUAGUU | SEQ ID NO: 102 |
| hsa-miR-126 | UCGUACCGUGAGUAAUAAUGCG | SEQ ID NO: 103 |
| hsa-miR-141 | UAACACUGUCUGGUAAAGAUGG | SEQ ID NO: 104 |
| hsa-miR-148a | UCAGUGCACUACAGAACUUUGU | SEQ ID NO: 105 |
| hsa-miR-148b | UCAGUGCAUCACAGAACUUUGU | SEQ ID NO: 106 |
| hsa-miR-361-5p | UUAUCAGAAUCUCCAGGGGUAC | SEQ ID NO: 107 |
| hsa-miR-625 | AGGGGGAAAGUUCUAUAGUCC | SEQ ID NO: 108 |
| hsa-miR-630 | AGUAUUCUGUACCAGGGAAGGU | SEQ ID NO: 109 |
| hsa-miR-766 | ACUCCAGCCCCACAGCCUCAGC | SEQ ID NO: 110 |

In particular, the miRNAs shown in Table 8 are present in higher quantity in the T_{H}17 lymphocytes than in the T_{H}1 or T_{H}2 lymphocytes or naive CD4⁺ T cells.

TABLE 8
List of the miRNAs overexpressed in the T_{H}17 lymphocytes
(compared to naive and other T helpers), with a minimum of
1.5 CT

| | | Sequence |
|---|---|---|
| miRNA | Target Sequence | Identifier |
| hsa-miR-126* | CAUUAUUACUUUUGGUACGCG | SEQ ID NO: 67 |
| hsa-miR-130a | CAGUGCAAUGUUAAAAGGGCAU | SEQ ID NO: 68 |
| hsa-miR-143* | GGUGCAGUGCUGCAUCUCUGGU | SEQ ID NO: 69 |
| hsa-miR-144* | GGAUAUCAUCAUAUACUGUAAG | SEQ ID NO: 70 |
| hsa-miR-145* | GGAUUCCUGGAAAUACUGUUCU | SEQ ID NO: 71 |
| hsa-miR-181a* | ACCAUCGACCGUUGAUUGUACC | SEQ ID NO: 72 |
| hsa-miR-188-3p | CUCCCACAUGCAGGGUUUGCA | SEQ ID NO: 73 |
| hsa-miR-193a-3p | AACUGGCCUACAAAGUCCCAGU | SEQ ID NO: 74 |
| hsa-miR-199a-3p | ACAGUAGUCUGCACAUUGGUUA | SEQ ID NO: 5 |
| hsa-miR-19b-2* | AGUUUUGCAGGUUUGCAUUUCA | SEQ ID NO: 75 |
| hsa-miR-202* | UUCCUAUGCAUAUACUUCUUUG | SEQ ID NO: 76 |
| hsa-miR-208 | AUAAGACGAGCAAAAAGCUUGU | SEQ ID NO: 77 |
| hsa-miR-220 | CCACACCGUAUCUGACACUUU | SEQ ID NO: 78 |
| hsa-miR-221 | AGCUACAUUGUCUGCUGGGUUUC | SEQ ID NO: 79 |
| hsa-miR-29b-1* | GCUGGUUUCAUAUGGUGGUUUAGA | SEQ ID NO: 80 |
| hsa-miR-29c* | UGACCGAUUUCUCCUGGUGUUC | SEQ ID NO: 81 |
| hsa-miR-302° | UAAGUGCUUCCAUGUUUUGGUGA | SEQ ID NO: 82 |
| hsa-miR-324-5p | CGCAUCCCCUAGGGCAUUGGUGU | SEQ ID NO: 83 |
| hsa-miR-331-5p | CUAGGUAUGGUCCCAGGGAUCC | SEQ ID NO: 84 |
| hsa-miR-33a* | CAAUGUUUCCACAGUGCAUCAC | SEQ ID NO: 85 |
| hsa-miR-376° | AUCAUAGAGGAAAAUCCACGU | SEQ ID NO: 86 |
| hsa-miR-450b-5p | UUUUGCAAUAUGUUCCUGAAUA | SEQ ID NO: 87 |
| hsa-miR-519e | AAGUGCCUCCUUUUAGAGUGUU | SEQ ID NO: 88 |
| hsa-miR-524-3p | GAAGGCGCUUCCCUUUGGAGU | SEQ ID NO: 89 |
| hsa-miR-548d-5p | AAAAGUAAUUGUGGUUUUUGCC | SEQ ID NO: 90 |
| hsa-miR-550* | UGUCUUACUCCCUCAGGCACAU | SEQ ID NO: 91 |
| hsa-miR-564 | AGGCACGGUGUCAGCAGGC | SEQ ID NO: 92 |
| hsa-miR-566 | GGGCGCCUGUGAUCCCAAC | SEQ ID NO: 93 |
| hsa-miR-582-5p | UUACAGUUGUUCAACCAGUUACU | SEQ ID NO: 94 |
| hsa-miR-587 | UUUCCAUAGGUGAUGAGUCAC | SEQ ID NO: 95 |
| hsa-miR-623 | AUCCCUUGCAGGGGCUGUUGGGU | SEQ ID NO: 96 |
| hsa-miR-628-3p | UCUAGUAAGAGUGGCAGUCGA | SEQ ID NO: 97 |
| hsa-miR-649 | AAACCUGUGUUGUUCAAGAGUC | SEQ ID NO: 98 |
| hsa-miR-672 | UGAGGUUGGUGUACUGUGUGUGA | SEQ ID NO: 99 |
| hsa-miR-708 | AAGGAGCUUACAAUCUAGCUGGG | SEQ ID NO: 100 |
| hsa-miR-98 | UGAGGUAGUAAGUUGUAUUGUU | SEQ ID NO: 101 |

In particular, the miRNAs shown in Table 9 are present in lower quantity in the T_{H}17 lymphocytes than in the T_{H}1 or T_{H}2 lymphocytes or naive CD4⁺ T cells.

TABLE 9
List of the miRNAs underexpressed in the T_{H}17 lymphocytes
(compared to naive and other T helpers),
with a minimum of 1.5 CT

| | | Sequence |
|---|---|---|
| miRNA | Target Sequence | Identifier |
| hsa-let-7a | UGAGGUAGUAGGUUGUAUAGUU | SEQ ID NO: 102 |
| hsa-miR-126 | UCGUACCGUGAGUAAUAAUGCG | SEQ ID NO: 103 |
| hsa-miR-141 | UAACACUGUCUGGUAAAGAUGG | SEQ ID NO: 104 |
| hsa-miR-148a | UCAGUGCACUACAGAACUUUGU | SEQ ID NO: 105 |
| hsa-miR-148b | UCAGUGCAUCACAGAACUUUGU | SEQ ID NO: 106 |
| hsa-miR-361-5p | UUAUCAGAAUCUCCAGGGGUAC | SEQ ID NO: 107 |
| hsa-miR-625 | AGGGGGAAAGUUCUAUAGUCC | SEQ ID NO: 108 |
| hsa-miR-630 | AGUAUUCUGUACCAGGGAAGGU | SEQ ID NO: 109 |
| hsa-miR-766 | ACUCCAGCCCCACAGCCUCAGC | SEQ ID NO: 110 |

### Example 4

The analysis were carried out naive CD4⁺ T lymphocytes isolated from peripheral blood of healthy donors.

Quantitative RT-PCR analysis, conducted as in example 1, showed the presence of 46 miRNAs, described in Table 10, which are present in higher or lower quantity in the naive CD4⁺ T lymphocytes than in the T_{H}1, T_{H}2 or T_{H}17 lymphocytes.

TABLE 10

| | | miRNA sequence |
|---|---|---|
| miRNA | Target sequence | number |
| hsa-let-7a | UGAGGUAGUAGGUUGUAUAGUU | SEQ ID NO: 102 |
| hsa-let-7b | UGAGGUAGUAGGUUGUGUGGUU | SEQ ID NO: 111 |
| hsa-let-7c | UGAGGUAGUAGGUUGUAUGGUU | SEQ ID NO: 112 |
| hsa-let-7e | UGAGGUAGGAGGUUGUAUAGUU | SEQ ID NO: 113 |
| hsa-let-7g | UGAGGUAGUAGUUUGUACAGUU | SEQ ID NO: 114 |
| hsa-miR-100 | AACCCGUAGAUCCGAACUUGUG | SEQ ID NO: 115 |
| hsa-miR-125b | UCCCUGAGACCCUAACUUGUGA | SEQ ID NO: 116 |
| hsa-miR-139-5p | UCUACAGUGCACGUGUCUCCAG | SEQ ID NO: 117 |
| hsa-miR-146b-5p | UGAGAACUGAAUUCCAUAGGCU | SEQ ID NO: 118 |
| hsa-miR-181a | AACAUUCAACGCUGUCGGUGAGU | SEQ ID NO: 119 |
| hsa-miR-181a-2* | ACCACUGACCGUUGACUGUACC | SEQ ID NO: 120 |
| hsa-miR-186 | CAAAGAAUUCUCCUUUUGGGCU | SEQ ID NO: 121 |
| hsa-miR-188-5p | CAUCCCUUGCAUGGUGGAGGG | SEQ ID NO: 122 |
| hsa-miR-191 | CAACGGAAUCCCAAAAGCAGCUG | SEQ ID NO: 123 |
| hsa-miR-193b | AACUGGCCCUCAAAGUCCCGCU | SEQ ID NO: 124 |
| hsa-miR-23a* | GGGGUUCCUGGGGAUGGGAUUU | SEQ ID NO: 125 |
| hsa-miR-26a | UUCAAGUAAUCCAGGAUAGGCU | SEQ ID NO: 126 |
| hsa-miR-30e* | CUUUCAGUCGGAUGUUUACAGC | SEQ ID NO: 127 |
| hsa-miR-335 | UCAAGAGCAAUAACGAAAAAUGU | SEQ ID NO: 128 |
| hsa-miR-342-5p | AGGGGUGCUAUCUGUGAUUGA | SEQ ID NO: 129 |
| hsa-miR-365 | UAAUGCCCCUAAAAAUCCUUAU | SEQ ID NO: 130 |
| hsa-miR-505 | CGUCAACACUUGCUGGUUUCCU | SEQ ID NO: 131 |
| hsa-miR-532-3p | CCUCCCACACCCAAGGCUUGCA | SEQ ID NO: 132 |
| hsa-miR-532-5p | CAUGCCUUGAGUGUAGGACCGU | SEQ ID NO: 133 |
| hsa-miR-576-3p | AAGAUGUGGAAAAAUUGGAAUC | SEQ ID NO: 134 |
| hsa-miR-579 | UUCAUUUGGUAUAAACCGCGAUU | SEQ ID NO: 135 |
| hsa-miR-629 | UGGGUUUACGUUGGGAGAACU | SEQ ID NO: 136 |
| hsa-miR-630 | AGUAUUCUGUACCAGGGAAGGU | SEQ ID NO: 109 |
| hsa-miR-645 | UCUAGGCUGGUACUGCUGA | SEQ ID NO: 137 |
| hsa-miR-92a-1* | AGGUUGGGAUCGGUUGCAAUGCU | SEQ ID NO: 138 |
| hsa-miR-99a | AACCCGUAGAUCCGAUCUUGUG | SEQ ID NO: 18 |
| hsa-miR-16-1* | CCAGUAUUAACUGUGCUGCUGA | SEQ ID NO: 139 |
| hsa-miR-18a | UAAGGUGCAUCUAGUGCAGAUAG | SEQ ID NO: 140 |
| hsa-miR-202 | AGAGGUAUAGGGCAUGGGAA | SEQ ID NO: 141 |
| hsa-miR-203 | GUGAAAUGUUUAGGACCACUAG | SEQ ID NO: 142 |
| hsa-miR-212 | UAACAGUCUCCAGUCACGGCC | SEQ ID NO: 143 |
| hsa-miR-27a* | AGGGCUUAGCUGCUUGUGAGCA | SEQ ID NO: 144 |
| hsa-miR-29a* | ACUGAUUUCUUUUGGUGUUCAG | SEQ ID NO: 145 |
| hsa-miR-330-3p | GCAAAGCACACGGCCUGCAGAGA | SEQ ID NO: 146 |
| hsa-miR-34a | UGGCAGUGUCUUAGCUGGUUGU | SEQ ID NO: 37 |
| hsa-miR-34a* | CAAUCAGCAAGUAUACUGCCCU | SEQ ID NO: 147 |
| hsa-miR-380* | UGGUUGACCAUAGAACAUGCGC | SEQ ID NO: 148 |
| hsa-miR-483-5p | AAGACGGGAGGAAAGAAGGGAG | SEQ ID NO: 149 |
| hsa-miR-518f | GAAAGCGCUUCUCUUUAGAGG | SEQ ID NO: 150 |
| hsa-miR-564 | AGGCACGGUGUCAGCAGGC | SEQ ID NO: 92 |
| hsa-miR-572 | GUCCGCUCGGCGGUGGCCCA | SEQ ID NO: 151 |

In particular, Table 11 shows the miRNAs present in higher quantity in the naive CD4⁺ T lymphocytes than in the T_{H}1, T_{H}2 or T_{H}17 lymphocytes.

TABLE 11
List of the miRNAs overexpressed in the naive CD4⁺ T
lymphocytes (compared to T_{H}1, T_{H}2 or T_{H}17 lymphocytes),
with a minimum of 1.5 CT

| | | miRNA sequence |
|---|---|---|
| miRNA | Target sequence | number |
| hsa-let-7a | UGAGGUAGUAGGUUGUAUAGUU | SEQ ID NO: 102 |
| hsa-let-7b | UGAGGUAGUAGGUUGUGUGGUU | SEQ ID NO: 111 |
| hsa-let-7c | UGAGGUAGUAGGUUGUAUGGUU | SEQ ID NO: 112 |
| hsa-let-7e | UGAGGUAGGAGGUUGUAUAGUU | SEQ ID NO: 113 |
| hsa-let-7g | UGAGGUAGUAGUUUGUACAGUU | SEQ ID NO: 114 |
| hsa-miR-100 | AACCCGUAGAUCCGAACUUGUG | SEQ ID NO: 115 |
| hsa-miR-125b | UCCCUGAGACCCUAACUUGUGA | SEQ ID NO: 116 |
| hsa-miR-139-5p | UCUACAGUGCACGUGUCUCCAG | SEQ ID NO: 117 |
| hsa-miR-146b-5p | UGAGAACUGAAUUCCAUAGGCU | SEQ ID NO: 118 |
| hsa-miR-181a | AACAUUCAACGCUGUCGGUGAGU | SEQ ID NO: 119 |
| hsa-miR-181a-2* | ACCACUGACCGUUGACUGUACC | SEQ ID NO: 120 |
| hsa-miR-186 | CAAAGAAUUCUCCUUUUGGGCU | SEQ ID NO: 121 |
| hsa-miR-188-5p | CAUCCCUUGCAUGGUGGAGGG | SEQ ID NO: 122 |
| hsa-miR-191 | CAACGGAAUCCCAAAAGCAGCUG | SEQ ID NO: 123 |
| hsa-miR-193b | AACUGGCCCUCAAAGUCCCGCU | SEQ ID NO: 124 |
| hsa-miR-23a* | GGGGUUCCUGGGGAUGGGAUUU | SEQ ID NO: 125 |
| hsa-miR-26a | UUCAAGUAAUCCAGGAUAGGCU | SEQ ID NO: 126 |
| hsa-miR-30e* | CUUUCAGUCGGAUGUUUACAGC | SEQ ID NO: 127 |
| hsa-miR-335 | UCAAGAGCAAUAACGAAAAAUGU | SEQ ID NO: 128 |
| hsa-miR-342-5p | AGGGGUGCUAUCUGUGAUUGA | SEQ ID NO: 129 |
| hsa-miR-365 | UAAUGCCCCUAAAAAUCCUUAU | SEQ ID NO: 130 |
| hsa-miR-505 | CGUCAACACUUGCUGGUUUCCU | SEQ ID NO: 131 |
| hsa-miR-532-3p | CCUCCCACACCCAAGGCUUGCA | SEQ ID NO: 132 |
| hsa-miR-532-5p | CAUGCCUUGAGUGUAGGACCGU | SEQ ID NO: 133 |
| hsa-miR-576-3p | AAGAUGUGGAAAAAUUGGAAUC | SEQ ID NO: 134 |
| hsa-miR-579 | UUCAUUUGGUAUAAACCGCGAUU | SEQ ID NO: 135 |
| hsa-miR-629 | UGGGUUUACGUUGGGAGAACU | SEQ ID NO: 136 |
| hsa-miR-630 | AGUAUUCUGUACCAGGGAAGGU | SEQ ID NO: 109 |
| hsa-miR-645 | UCUAGGCUGGUACUGCUGA | SEQ ID NO: 137 |
| hsa-miR-92a-1* | AGGUUGGGAUCGGUUGCAAUGCU | SEQ ID NO: 138 |
| hsa-miR-99a | AACCCGUAGAUCCGAUCUUGUG | SEQ ID NO: 18 |

In particular, Table 12 shows the miRNAs present in lower quantity in the naive CD4⁺ T lymphocytes than in the T_{H}1, T_{H}2 or T_{H}17 lymphocytes.

TABLE 12
List of the miRNAs underexpressed in the naive CDC T
lymphocytes (compared to T_{H}1, T_{H}2 or T_{H}17 lymphocytes),
with a minimum of 1.5 CT

| | | miRNA sequence |
|---|---|---|
| miRNA | Target sequence | number |
| hsa-miR-16-1* | CCAGUAUUAACUGUGCUGCUGA | SEQ ID NO: 139 |
| hsa-miR-18a | UAAGGUGCAUCUAGUGCAGAUAG | SEQ ID NO: 140 |
| hsa-miR-202 | AGAGGUAUAGGGCAUGGGAA | SEQ ID NO: 141 |
| hsa-miR-203 | GUGAAAUGUUUAGGACCACUAG | SEQ ID NO: 142 |
| hsa-miR-212 | UAACAGUCUCCAGUCACGGCC | SEQ ID NO: 143 |
| hsa-miR-27a* | AGGGCUUAGCUGCUUGUGAGCA | SEQ ID NO: 144 |
| hsa-miR-29a* | ACUGAUUUCUUUUGGUGUUCAG | SEQ ID NO: 145 |
| hsa-miR-330-3p | GCAAAGCACACGGCCUGCAGAGA | SEQ ID NO: 146 |
| hsa-miR-34a | UGGCAGUGUCUUAGCUGGUUGU | SEQ ID NO: 37 |
| hsa-miR-34a* | CAAUCAGCAAGUAUACUGCCCU | SEQ ID NO: 147 |
| hsa-miR-380* | UGGUUGACCAUAGAACAUGCGC | SEQ ID NO: 148 |
| hsa-miR-483-5p | AAGACGGGAGGAAAGAAGGGAG | SEQ ID NO: 149 |
| hsa-miR-518f | GAAAGCGCUUCUCUUUAGAGG | SEQ ID NO: 150 |
| hsa-miR-564 | AGGCACGGUGUCAGCAGGC | SEQ ID NO: 92 |
| hsa-miR-572 | GUCCGCUCGGCGGUGGCCCA | SEQ ID NO: 151 |

### Example 5

The analysis were carried out on 13 subjects with psoriasis. The tissue analyzed consisted in peripheral blood and the experimental control was represented by the peripheral blood of healthy donors.

The total RNA was extracted from 70 μl of serum using the mirVana™ miRNA Isolation Kit (Cat# AM1561— Ambion). Synthetic RNA ath-miRl59a, Arabidopsis thaliana microRNA not expressed in man, was added as a quantitative normalizer (3 fmoles per aliquot of serum). An aliquot of the sample (3 μL of the total 50 μL of extracted RNA) was submitted to a reverse transcription reaction conducted using the TaqMan® MicroRNA Reverse Transcription kit in the presence of a solution of MgCl₂ 5 mM (Part no. 4366597— Applied Biosystems). Primers specific for hsa-miR564, specifically expressed by the T_{H}17 lymphocytes, and for ath-miRl59a were used as primers for the reverse transcription (Applied Biosystem Assay ID 001531 and Assay ID 000338). Final reaction volume (μL): 15.

Incubation Conditions for a Reaction Cycle:

16° C. 30 min

42° C. 30 min

85° C. 5 min

4° C. ∞

(for 40 cycles)

The same volume of cDNA produced from serum of psoriatic patients and healthy donors was used for the real-time PCR reaction. The reaction was conducted using TaqMan Universal PCR Master Mix, No Amperase UNG, 2×(Part No: 4326614—Applied Biosystems) in final 20 μL with primers and a Taqman probe specific for hsa-miR564 and ath-miRl59a (Applied Biosystem Assay ID 001531 and Assay ID 000338).

The internal control ath-miRl59a can be used to calculate relative gene expression. The relative expression of each miRNA can be calculated using the equation 2^{-Δct}, where ΔCt=(Ct miRNA)−(Ct ath-miR159a).

FIG. 5 shows the values of the RT-PCR analysis.

In particular, the values of hsa-miR-564 (Seq ID NO: 92), which is expressed to the largest degree in the CD4⁺ T_{H}17 lymphocyte population, show an increase in the blood of patients with psoriatic arthritis compared to the controls (healthy donors). An analogous analysis conducted on a control miRNA (hsa-miR-200) shows no significant differences between patients with psoriasis and healthy donors.

### Example 6

For the purpose of analyzing miRNA expression in human primary lymphocytes, 17 subpopulations of T cells, B cells and NK cells were used.

In particular, the subpopulations analyzed were: naive CD4⁺ T, CD4⁺ T_{H}1, CD4⁺ T_{H}2, CD4⁺ T_{H}17, CD4⁺ T_{reg}, memory CD4⁺, CD4⁺ EM, CD4⁺ CM, CD4⁺ EMRA, CD8⁺ naive, CD8⁺ EM, CD8⁺ CM, CD8⁺ EMRA, CD5⁺ B, naive B, memory B and NK.

The lymphocyte subpopulations were purified by FACS, exploiting the fact that they express specific surface markers. In particular, the cell subpopulations were obtained from peripheral blood mononuclear cell samples (PBMCs) taken from 3 of 6 healthy donor individuals.

242 miRNAs expressed in a characteristic manner were identified in the cell subpopulations analyzed.

The expression of these miRNAs was analyzed by unsupervised hierarchical clustering and the results showed a clear categorization of the samples of NK cells, CD4⁺ T lymphocytes, CD8⁺ T lymphocytes and B lymphocytes, which reflects the phenotypic classification of the subpopulations. Furthermore, through this approach it was possible to identify miRNAs which had never been associated with the lymphocyte cell subpopulations examined.

Comparing the miRNAs expressed by the 17 cell subpopulations characterized by an expression level at least 3 times higher than that of a some subpopulation (via one-way ANOVA—p<0.01), 29 miRNAs were identified which show a specific expression of the subpopulation (FIG. 6).

The expression of SEQ ID NO: 116 (hsa-miR-125b), SEQ ID NO: 124 (hsa-miR-193b) and SEQ ID NO: 122 (hsa-miR-188-5p) had never been associated in a selective manner with the naive CD4⁺ T population before now.

The expression of SEQ ID NO: 3 (hsa-miR-381) is selective for CD4⁺ T_{H}1 cells.

The miRNAs which are expressed in a differential manner in the various states of differentiation of the naive CD4⁺ T helper cell line, i.e. the memory cells or T_{H}1, T_{H}2 and T_{H}17 lymphocytes (FIG. 7A), were moreover identified.

The miRNAs overexpressed in the naive CD4⁺ T cells as compared to the T_{H}1, T_{H}2 and T_{H}17 lymphocytes are shown in table 13.

TABLE 13

| | | Sequence |
|---|---|---|
| miRNA name | miRNA sequence | number |
| hsa-miR-146b-5p | UGAGAACUGAAUUCCAUAGGCU | SEQ ID NO: 118 |
| hsa-miR-26a | UUCAAGUAAUCCAGGAUAGGCU | SEQ ID NO: 126 |
| hsa-let-7e | UGAGGUAGGAGGUUGUAUAGUU | SEQ ID NO: 113 |
| hsa-miR-191 | CAACGGAAUCCCAAAAGCAGCUG | SEQ ID NO: 123 |
| hsa-miR-188-5p | CAUCCCUUGCAUGGUGGAGGG | SEQ ID NO: 122 |
| has-let-7g | UGAGGUAGUAGUUUGUACAGUU | SEQ ID NO: 114 |
| hsa-let-7b | UGAGGUAGUAGGUUGUGUGGUU | SEQ ID NO: 111 |
| hsa-miR-186 | CAAAGAAUUCUCCUUUUGGGCU | SEQ ID NO: 121 |
| hsa-miR-193b | AACUGGCCCUCAAAGUCCCGCU | SEQ ID NO: 124 |
| hsa-miR-181a | AACAUUCAACGCUGUCGGUGAGU | SEQ ID NO: 119 |
| hsa-miR-125b | UCCCUGAGACCCUAACUUGUGA | SEQ ID NO: 116 |
| hsa-miR-99a | AACCCGUAGAUCCGAUCUUGUG | SEQ ID NO: 18 |
| hsa-miR-532-3p | CCUCCCACACCCAAGGCUUGCA | SEQ ID NO: 132 |
| hsa-miR-100 | AACCCGUAGAUCCGAACUUGUG | SEQ ID NO: 115 |
| hsa-miR-365 | UAAUGCCCCUAAAAAUCCUUAU | SEQ ID NO: 130 |
| hsa-miR-532-5p | CAUGCCUUGAGUGUAGGACCGU | SEQ ID NO: 133 |
| hsa-let-7c | UGAGGUAGUAGGUUGUAUGGUU | SEQ ID NO: 112 |
| hsa-miR-335 | UCAAGAGCAAUAACGAAAAAUGU | SEQ ID NO: 128 |
| hsa-miR-645 | UCUAGGCUGGUACUGCUGA | SEQ ID NO: 137 |
| hsa-miR-576-3p | AAGAUGUGGAAAAAUUGGAAUC | SEQ ID NO: 134 |

The miRNAs underexpressed in the naive CD4⁺ T cells compared to the T_{H}1, T_{H}2 and T_{H}17 lymphocytes are shown in table 14.

TABLE 14

| miRNA name | miRNA sequence | Sequence number |
|---|---|---|
| hsa-miR-27a* | AGGGCUUAGCUGCU | SEQ ID NO: 144 |
| | UGUGAGCA | |
| has-miR-597 | UGUGUCACUCGAUG | SEQ ID NO: 152 |
| | ACCACUGU | |
| hsa-miR-483-5p | AAGACGGGAGGAAA | SEQ ID NO: 149 |
| | GAAGGGAG | |
| hsa-miR-885-5p | UCCAUUACACUACC | SEQ ID NO: 58 |
| | CUGCCUCU | |
| has-miR-638 | AGGGAUCGCGGGCG | SEQ ID NO: 153 |
| | GGUGGCGGCCU | |

The miRNAs listed in table 15 are differentially expressed in the T_{H}1 lymphocytes. In particular, SEQ ID NO: 3 (hsa-miR-381) and SEQ ID NO: 1 (hsa-miR-135b) are overexpressed, whereas SEQ ID NO: 18 (hsa-miR-99a) and SEQ ID NO: 9 (hsa-miR-425*) are underexpressed.

TABLE 15

| miRNA name | miRNA sequence | Sequence number |
|---|---|---|
| hsa-miR-381 | UAUACAAGGGCAAGCUCUCUGU | SEQ ID NO: 3 |
| hsa-miR-135b | UAUGGCUUUUCAUUCCUAUGUGA | SEQ ID NO: 1 |
| hsa-miR-99a | AACCCGUAGAUCCGAUCUUGUG | SEQ ID NO: 18 |
| hsa-miR-425* | AUCGGGAAUGUCGUGUCCGCCC | SEQ ID NO: 9 |

The miRNAs listed in table 16 are differentially expressed in the T_{H}17 lymphocytes. In particular, SEQ ID NO: 67 (hsa-miR-126*) is overexpressed, whereas SEQ ID NO: 105 (hsa-miR-148a) is underexpressed.

TABLE 16

| miRNA name | miRNA sequence | Sequence number |
|---|---|---|
| hsa-miR-126* | CAUUAUUACUUUUG | SEQ ID NO: 67 |
| | GUACGCG | |
| hsa-miR-148a | UCAGUGCACUACAG | SEQ ID NO: 105 |
| | AACUUUGU | |

The miRNAs listed in table 17 are overexpressed in the T_{H}2 lymphocytes.

TABLE 17

| miRNA name | miRNA sequence | Sequence number |
|---|---|---|
| hsa-miR-190b | UGAUAUGUUUGAUA | SEQ ID NO: 27 |
| | UUGGGUU | |
| hsa-miR-215 | AUGACCUAUGAAUU | SEQ ID NO: 32 |
| | GACAGAC | |
| hsa-miR-551b* | GAAAUCAAGCGUGG | SEQ ID NO: 48 |
| | GUGAGACC | |
| hsa-miR-626 | AGCUGUCUGAAAAU | SEQ ID NO: 154 |
| | GUCUU | |

For the purpose of validating the data regarding the specific expression of the groups of miRNAs (defined “signature”) in the naive CD4⁺ T cells and in the T_{H}1, T_{H}2 and T_{H}17 lymphocytes, the variation in their expression was evaluated through in vitro experiments based on activation of the naive cells. The activation of naive cells induces their differentiation into T_{H}1, T_{H}2 and T_{H}17 lymphocytes.

The expression of the miRNAs of interest was determined at different times following activation of the naive cells (see FIG. 7B).

The expression of 19 of the 20 miRNAs highly expressed in the naive cells is extinguished after cell activation, whereas there is an increase in the expression of 4 of the 5 miRNAs highly expressed in the memory cells.

Materials and Methods

Purification of the subpopulations of primary lymphocytes. Buffy-coat samples from healthy blood donors were supplied by Ospedale Maggiore of Milan and the peripheral blood mononuclear cells were isolated by Ficoll gradient centrifugation.

The primary lymphocytes from human blood were purified (>95% of purity) by FACS using different combinations of surface markers.

The NK cells were selected as CD56⁺-CD3⁻ cells.

The subpopulations of naive B cells and memory B cells were isolated for the expression of CD19, CD5 and CD27.

The subpopulations of CD4⁺ cells, naive CD8⁺ cells, central memory and effector memory T cells were isolated for the expression of CD45RA, CD45R0 and CCR7.

The subpopulations of T_{H}1, T_{H}2 and T_{H}17 lymphocytes were isolated from the total population of memory CD4⁺ T cells (CD45RA⁻, CD45R0⁺) respectively as (CXCR3⁺, CCR6⁻, CD161⁻), (CRTH2⁺, CXCR3⁻) and (CXCR3⁻, CCR6⁺, CD161⁺) cells.

For the in vitro differentiation experiments, the naive CD4⁺ T cells were purified by negative immunomagnetic selection and subsequently stimulated with the anti-CD3 and anti-CD28 antibodies bound to a plastic substrate.

After stimulation, IL-2 was added at a concentration of 20 IU/ml. In order to verify the production of interferon gamma (INF-γ), the cells were stimulated for 4 hours with PMA and ionomycin (after 2 hours BFA is added) and after that the presence of INF-γ was verified using a PB-conjugated anti-INF-γ antibody.

IL-3 production by the cells was verified using a PE-conjugated anti-IL3 antibody.

In parallel, the cells were collected at different time intervals for extraction of the total RNA and the miRNA profile was analyzed by means of TaqMan Low Density assays (TLDAs).

The gene expression of the entire transcriptome was determined in the naive CD4⁺ cells and memory T cells by Illumina Direct Hybridization Assay, in accordance with the standard procedure.

The total RNA was isolated, checked for quality and then quantized.

For each sample of naive CD4⁺ cells and memory T cells, 500 ng of total RNA was reverse transcribed using the Illumina TotalPrep RNA Amplification kit (Ambion) and the cRNAs were generated after 14 hours of in vitro transcription.

Washing, staining and hybridization were carried out in accordance with the standard Illumina protocol.

In particular, for each sample, 750 ng of cRNA was hybridized to an Illumina Human HT-12 v3 Expression BeadChip array in a final volume of 15 μl.

Hybridization and scanning were performed using the Illumina iScan System in accordance with the instructions provided and the data obtained were processed with BeadStudio v.3.

The arrays were normalized without background subtraction and the mean value of the signals was calculated based on the gene level data for the genes whose determination p-value was lower than 0.001 in at least one of the two cohorts considered (naive CD4⁺ and memory T cells).

1. A method for monitoring the immune system of an individual, comprising the steps of:
a) measuring the expression level of at least one miRNA gene product selected from among SEQ ID NO: 1-154 or combinations thereof; and
b) comparing said measured expression level with a reference level,
wherein said measuring of the expression level of at least one miRNA gene product is carried out in an isolated sample of peripheral blood or biological fluid.

2. The method according to claim 1, wherein said individual is affected by a pathological condition caused by or associated with a dysfunction of said immune system, or said individual undergoes a vaccination.

3. The method according to claim 1, for the diagnosis, prognosis or prevention of a pathological condition caused by or associated with a dysfunction of said immune system, or for evaluating the risk of the functionality of said immune system being compromised, or for monitoring the effectiveness of a therapeutic treatment for said pathological condition caused by or associated with a dysfunction of said immune system.

4. The method claim 1, wherein said at least one miRNA gene product is expressed by lymphocyte populations of the immune system, preferably by T lymphocytes.

5. The method according to claim 4, wherein said T lymphocytes are T helper lymphocytes expressing the protein CD4, preferably they are naive CD4⁺ T, T_{H}1, T_{H}2 or T_{H}17 lymphocytes or combinations thereof.

6. The method according to claim 1, wherein said pathological conditions caused by or associated with an immune system dysfunction are selected from among immunodeficiencies, neoplasia of the immune system, immune-mediated pathologies, said immune-mediated pathologies preferably being allergic conditions or autoimmune pathologies.

7. The method according to claim 6, wherein said autoimmune pathologies are selected from among: systemic lupus erythematosus, rheumatoid arthritis, ankylosing spondylitis, multiple sclerosis, type 1 diabetes mellitus and psoriatic arthritis.

8. The method according to claim 1, wherein said at least one miRNA gene product is selected from among: SEQ ID NO: 1-3, 5, 10, 14, 18, 19-58, 67-101, 102, 109, 111-138 and 154; preferably it is selected from among: SEQ ID NO: 1, 3, 18, 27, 32-33, 48, 58, 67, 79, 84, 92, 111-116, 118-119, 121-124, 126, 128, 130, 132-134, 137 and 154.

9. The method according to claim 1, wherein said at least one miRNA gene product is selected from among: SEQ ID NO: 4-18, 37, 92, 59-66, 102-110 and 139-153; more preferably it is selected from among: SEQ ID NO: 9, 18, 144, 149, 152 and 153.

10. The method according to claim 1, wherein said at least one miRNA gene product is selected from among: SEQ ID NO: 18, 37, 92, 102, 109 and 111-153 and is overexpressed or underexpressed in a subject affected by a pathological condition caused by an immune system dysfunction compared to a control or in a subject on whom a vaccination was performed compared to a control; preferably said at least one miRNA gene product is overexpressed or underexpressed by naive CD4⁺ T lymphocytes.

11. The method according to claim 1, wherein said at least one miRNA gene product is selected from among: SEQ ID NO: 18, 58, 111-116, 118, 119, 121-124, 126, 128, 130, 132-134, 137, 144, 149 and 152-153, said at least one miRNA gene product being selected from among: SEQ ID NO: 18, 111-116, 118, 119, 121-124, 126, 128, 130, 132-134, 137 overexpressed in a subject affected by a pathological condition caused by or associated with an immune system dysfunction compared to a control, or in a sample of a subject on whom a vaccination was performed compared to a control; and/or said at least one miRNA gene product being selected from among: SEQ ID NO: 58, 144, 149, 152-153 underexpressed in a subject affected by a pathological condition caused by or associated with an immune system dysfunction compared to a control, or in a sample of a subject on whom a vaccination was performed compared to a control.

12. The method according to claim 11, wherein said at least one miRNA gene product is overexpressed and/or underexpressed by naive CD4⁺ T lymphocytes.

13. The method according to claim 1, wherein said at least one miRNA gene product is selected from among: SEQ ID NO: 1-18 and is overexpressed or underexpressed in a subject on whom a vaccination was performed compared to a control, preferably said at least one miRNA gene product is overexpressed or underexpressed by T_{H}1 lymphocytes.

14. The method according to claim 1, wherein said at least one miRNA gene product is selected from among: SEQ ID NO: 1, 3, 9 and 18, said at least one miRNA gene product being selected from among SEQ ID NO: 1 and 3 overexpressed in a subject on whom a vaccination was performed compared to a control; and/or said at least one miRNA gene product being selected from among: SEQ ID NO: 9 and 18 underexpressed in a subject on whom a vaccination was performed compared to a control.

15. The method according to claim 14, wherein said at least one miRNA gene product is overexpressed and/or underexpressed by T_{H}1 lymphocytes.

16. The method according to claim 1, wherein said at least one miRNA gene product is selected from among: SEQ ID NO: 10, 14 and 19-66 and is overexpressed or underexpressed in a subject affected by an allergy compared to a control; preferably said at least one miRNA gene product is overexpressed or underexpressed by T_{H}2 lymphocytes.

17. The method according to claim 1, wherein said at least one miRNA gene product is selected from among: SEQ ID NO:27, SEQ ID NO: 32, SEQ ID NO: 48 and SEQ ID NO: 154, said at least one miRNA gene product being selected from among: SEQ ID NO: 27, SEQ ID NO: 32, SEQ ID NO: 48 and SEQ ID NO: 154 overexpressed in a subject affected by an allergy compared to a control.

18. The method according to claim 17, wherein said at least one miRNA gene product is overexpressed and/or underexpressed by T_{H}2 lymphocytes.

19. The method according to claim 1, wherein said at least one miRNA gene product is selected from among: SEQ ID NO: 5, 67-110 and is overexpressed or underexpressed in a subject affected by an autoimmune disease compared to a control; preferably said at least one miRNA gene product is overexpressed or underexpressed by T_{H}17 lymphocytes.

20. The method according to claim 1, wherein said at least one gene product is selected from among: SEQ ID NO: 67 and SEQ ID NO: 105, SEQ ID NO: 67 being overexpressed in a subject affected by an autoimmune disease compared to a control; and/or SEQ ID NO: 105 being underexpressed in a subject affected by an autoimmune disease compared to a control.

21. The method according to claim 20, wherein said at least one miRNA gene product is overexpressed and/or underexpressed by T_{H}17 lymphocytes.

22. The method according to claim 1, wherein said sample of peripheral blood is selected from among whole blood, peripheral blood mononuclear cells, serum or plasma; said sample of biological fluid is selected from between urine or saliva.

23. (canceled)

24. A pharmaceutical composition comprising a pharmaceutically acceptable carrier and at least one isolated miRNA gene product according to claim 1 and/or a nucleic acid complementary thereto.

25. The composition according to claim 24, for use in treating a pathological condition caused by an immune system dysfunction.
